(19) United States
(12) Patent Application Publication
(10) Pub. No.: US 2026/0108574 A1
(43) Pub. Date: Apr. 23, 2026

(54) HERBAL COMPOSITION FOR TREATING METABOLIC DISORDERS IN MAMMALS

(71) Applicants: ANIRUDH MURTHY RANGA, Mysore (IN); SAMEER KAMALAKAR AKOLKAR, Aurangabad (IN); MELUKOTE SRINIVASA SHETTY MADHUKUMAR, Mysore (IN)

(72) Inventors: ANIRUDH MURTHY RANGA, Mysore (IN); SAMEER KAMALAKAR AKOLKAR, Aurangabad (IN); MELUKOTE SRINIVASA SHETTY MADHUKUMAR, Mysore (IN)

(73) Assignee: M/S NATURAL AND ESSENTIAL OILS PRIVATE LIMITED, Mysore (IN)

(21) Appl. No.: 19/360,002

(22) Filed: Oct. 16, 2025

(30) Foreign Application Priority Data
Oct. 17, 2024 (IN) 202441054498

(51) Int. Cl. A61K 36/48; A61K 36/21; A61K 36/54; A61P 3/04; A61P 3/06; A61P 25/22

(52) U.S. Cl. CPC A61K 36/48; A61K 36/21; A61K 36/54; A61P 3/04; A61P 3/06; A61P 25/22

(57) ABSTRACT

A herbal composition for the treatment of a variety of metabolic disorders in mammals, especially human beings, is disclosed. A method of preparing the herbal composition is also disclosed. The herbal composition treats and alleviates a plurality of metabolic disorders, including central obesity, high blood pressure levels, high blood sugar levels, stress-induced fatigue, and type 2 diabetes. The herbal composition comprises the extracts of Dolichos lablab, Achyranthes aspera, and at least one of Cinnamomum zeylanicum and Cinnamomum cassia. The aforementioned extracts are present in the herbal composition at a predetermined weight range. The aforementioned extracts are standardized to contain active compounds, including proanthocyanidins, saponins, polyphenols, and tannins, in a predetermined concentration and in predetermined purity levels. The herbal composition causes an enhancement of adiponectin levels and high-density lipoprotein (HDL) levels, and a reduction in the accumulation of adipose tissue, total cholesterol levels, triglyceride levels, and low-density lipoprotein (LDL) levels.

This application claims priority from copending provisional application INDIA 202441054498, filed 17 Oct. 2024, the entire disclosure of which is hereby incorporated by reference.

The present disclosure relates to compositions that treat and alleviate metabolic disorders in mammals. More particularly, the present disclosure is directed to the field of natural remedies for metabolic disorders and the development of compositions that cause no serious side effects while treating and alleviating metabolic disorders and the related symptoms in mammals.

### BACKGROUND

Mammals, especially human beings, are affected by a variety of metabolic disorders, including dyslipidemia, stress-induced fatigue, pre-diabetes, high blood sugar levels, elevated blood pressure levels, and abdominal obesity (central obesity or visceral obesity). Among these metabolic disorders, abdominal obesity, elevated blood pressure levels, and high blood sugar levels are gaining prominence, for these metabolic disorders, if ignored in early stages, are likely to metamorphose into life-threatening diseases, including chronic diabetes and cardiac arrest. Further, these metabolic disorders are also known to significantly influence the dietary habits of human beings and the way human beings go about their daily lives.

Typically, abdominal obesity, also referred to as central obesity or visceral obesity, involves excess accumulation of fat in the abdominal region, particularly around internal organs such as the liver, pancreas, and intestines. Typically, abdominal obesity inhibits a metabolically (biologically) active type of fat distribution and is characterized as a metabolic disorder rather than general body fat. Also, abdominal obesity causes the release of pro-inflammatory cytokines (tumor necrosis factor-alpha and Interleukin-6) and hormones that disrupt human body metabolism. Further, central obesity overburdens the liver by causing a direct release of free fatty acids (FFA) into the portal vein of the human body. Further, central obesity also brings about a reduction in the synthesis of adiponectin, a protective hormone with anti-inflammatory properties, that improves glucose and lipid metabolism and insulin sensitivity in the human body. Lower levels of adiponectin, in turn, can lead to insulin resistance, metabolic syndrome (MetS), and an increased risk of the onset of cardiovascular diseases (CVD).

Chronic stress, hormonal, physical as well as cognitive, along with insulin resistance, has been established as a major factor that contributes significantly to the onset of central obesity in the human body. Typically, central obesity encourages and enhances insulin resistance in the human body and, in turn, contributes to the onset of type 2 diabetes. Central obesity also contributes to the onset of dyslipidemia, which, in turn, is characterized by abnormally high levels of high-density lipoprotein (HDL) and triglycerides. Central obesity acts as a contributory factor for hypertension and non-alcoholic fatty liver disease (NAFLD). Central obesity is also known to enhance the likelihood of heart attacks, cardiac arrest, and certain types of cancers, including colon cancer and pancreatic cancer.

Typically, elevated blood pressure refers to a health condition where the force of the blood against the artery walls is consistently higher than normal in the human body. If not cured or alleviated during the early stages, elevated blood pressure typically leads to high blood pressure (hypertension) and an array of cardiovascular diseases, including heart attack, permanent damage to blood-carrying vessels, and stroke. It has been clinically established that central obesity and chronic stress, hormonal, physical and cognitive, act as contributory factors for elevated blood pressure and elevated blood sugar levels. It has been clinically established that elevated blood pressure is caused by other health-related factors such as hormonal imbalance, kidney dysfunction, and physical inactivity.

Typically, high blood sugar levels (hyperglycemia) occur in the human body when the amount of glucose in the blood stream (of the human body) exceeds pre-defined normal levels. High blood sugar levels, if left untreated during the early stages, often metamorphose into pre-diabetes and subsequently diabetes mellitus. It has been observed that stress is also one of the contributing factors for the onset of high blood sugar levels in the human body. It has been clinically established that central obesity, hormonal stress, lowered insulin production rates, and insulin resistance contribute significantly to the onset of high blood sugar levels in the human body.

Further, while stress, either hormonal, physical, or cognitive, is not a metabolic disorder in itself, it has been clinically established that chronic stress, regardless of its type, contributes to a variety of metabolic disorders, including insulin resistance, central obesity, high blood sugar levels, high blood pressure levels, and dyslipidemia.

It has also been clinically established that central obesity, in addition to being a major and life-threatening metabolic disorder, also contributes significantly to the onset of high blood pressure (hypertension) and high blood sugar levels (hyperglycemia) in the human body. Typically, a larger body with a substantial amount of fat accumulated around the belly region requires the heart to work harder to supply blood to all parts of the body and is thus likely to suffer from damage to kidneys that regulate blood pressure. Further, damage to kidneys could also cause a variety of renal diseases, including chronic kidney disease (CKD) and acute kidney injury. Previously performed studies have established a clear connection between central obesity and hypertension, and have also labelled central obesity as the major cause of hypertension and a variety of renal diseases.

Furthermore, it has also been established that central obesity causes insulin resistance and thus increases the risk of the human body developing high blood sugar levels, where the human body's cells do not respond effectively to insulin, which, in turn, regulates blood sugar levels. High blood sugar levels in the human body contribute to the onset of type 2 diabetes, a chronic and a life-threating condition caused by the human body's inability to either use the naturally produced insulin to effectively regulate blood sugar levels (insulin sensitivity) or produce insulin sufficient enough to absorb the requisite amount of blood sugar from the blood stream and convert the blood sugar into energy. Further, the inability of the human body to produce enough insulin to absorb the requisite amount of blood sugar from the bloodstream also causes fatigue, which, if untreated in the earlier stages, may turn chronic.

Further, when the human body experiences stress (physical or cognitive) for a prolonged period of time, it activates the hypothalamic-pituitary-adrenal (HPA) axis and the sympathetic nervous system, which, in turn, enhances the release of stress hormones, viz., cortisol (from the adrenal cortex), adrenaline, and noradrenaline (both from the adrenal medulla). While the human body adapts to the release of the above-identified stress hormones (cortisol, adrenaline, and noradrenaline) in the short term, long term effects associated with the continued release of stress hormones are often harmful and act as the catalyst for a multitude of metabolic disorders, including insulin resistance, visceral fat accumulation, hypertension, dyslipidemia, hyperglycemia, and type 2 diabetes. It has been clinically established that chronic stress and the consequential prolonged release of stress-related hormones enhances glucose production, thus leading to high blood sugar levels, makes muscle cells and fat cells (in the body) insulin resistant, stimulates fat storage in the abdomen, thus contributing significantly to central obesity, enhances the blood pressure, thus leading to high blood pressure levels in the long term, and alters the gut-brain axis, thus triggering significant changes in the appetite and a craving for high-calorie foods, which, in turn, promote central obesity. Stress acts as both a trigger and an amplifier of metabolic disorders. As described above, chronic activation of stress hormones (cortisol, adrenaline, and noradrenaline) causes metabolic dysregulation and contributes to the onset of diseases such as diabetes, central obesity, hyperglycemia, and hypertension, among others.

From the aforementioned paragraphs, it is apparent that chronic stress (either hormonal, physical, or cognitive) is one of the major contributing factors for a multitude of metabolic disorders in the human body. Chronic stress, as described above, both triggers and amplifies a multitude of metabolic disorders in the human body, including central obesity, reduction in insulin sensitivity, insulin resistance, high blood pressure levels, high blood sugar levels, hypertension, and dyslipidemia, among others.

However, even when human beings manage to control their stress levels or significantly bring down their stress levels, the metabolic disorders introduced into the human body due to chronic stress, especially central obesity and type 2 diabetes, do not wane without medical intervention and extensive treatment. Moreover, metabolic disorders such as central obesity, stress-induced fatigue, diabetes, and visceral fat accumulation are known to be recurring and are highly resistant to diet changes and exercise regimen.

While stress is one of the major contributory factors for the onset of central obesity in human beings, poor nutrition levels, poor diet choices, and a sedentary lifestyle with no regular cardiovascular activities and exercises also promotes the rate at which the human body accumulates fat, especially around the abdomen, and thus exacerbates central obesity. Central obesity typically metamorphoses into a state of chronic inflammation, especially around the abdomen in the human body, and is likely to cause abnormal hormonal responses and immune system reactions. Central obesity, if left untreated during the earlier stages, eventually brings about a systemic metabolic dysregulation in the human body. Typically, the causes of central obesity are manifold and involve genetics, environmental factors, socio-economic factors, and behavioral factors. These factors, when not addressed efficiently and effectively through therapeutic means in the early stages, not only contribute to the onset of central obesity but also to the persistence of central obesity.

It is well-established that a human body suffering from central obesity also exhibits adipose tissue dysfunctionality, which, in turn, creates a metabolic and inflammatory imbalance in the human body, alters cell function, increases inflammation, and impairs the human body's ability to release the stored fat. Furthermore, adipose tissue dysfunctionality in obese individuals (obese human bodies) also brings about a substantial increase in the secretion of pro-inflammatory proteins, including interleukin-6 (IL-6), tumor necrosis factor-alpha (TNF-α), C-reactive protein (CRP), and interleukin-18 (IL-18). The metabolic and inflammatory imbalance in obese individuals also leads to insulin resistance, persistent central obesity, systemic inflammation, and increases the risk of renal diseases, hypertension, atherosclerosis, type 2 diabetes, and a variety of cardiovascular diseases, including heart attack, stroke, and cardiac arrest.

Further, several drug-based treatments for the alleviation of the above-identified metabolic disorders and the symptoms thereof, especially central obesity, stress-induced fatigue, and type 2 diabetes. Most of the known therapeutic approaches for treating the above-identified metabolic disorders are harsh on the gastrointestinal (GI) tract. Further, most of the medications are not easily absorbed by the target body cells (in the human body). Further, most of the prior art medications cause bloating, formation of gas, liver stress, and significant side-effects when combined with other antidiabetic medications, antihypertensive medications, and anticoagulant medications. Further, herbal therapeutic compositions used for treating metabolic disorders lack standardization, viz., the individual herbs used in such compositions vary drastically in terms of purity and potency. Furthermore, most of the prior art herbal compositions are also plagued by the delayed onset of therapeutic effects and benefits. Furthermore, most of the herbal compositions are not backed by appropriate clinical trials and lack therapeutic efficacy data.

One of the common medications used for treating obesity and diabetes (both being commonly known metabolic disorders) is Semaglutide™. Semaglutide mimics the natural hormone glucagon-like peptide-1 (GLP-1) and helps regulate blood sugar levels by increasing insulin secretion and reducing glucagon in the human body. Further, Semaglutide also promotes weight loss by increasing the feelings of stomach fullness and by reducing appetite, and thus causing the recipient human being to eat less. However, not only is Semaglutide expensive, but the use of Semaglutide also causes gastrointestinal side effects such as nausea, vomiting, diarrhea, constipation, and dehydration. Some of the serious risks associated with the prolonged use of Semaglutide include diabetic retinopathy, acute pancreatitis, acute kidney injury, and gallbladder disorders. In addition, Semaglutide also causes injection site reactions (when consumed in the form of an injection) and hypoglycemia (low blood sugar levels). Moreover, it has been established that the prolonged use of Semaglutide may also cause thyroid C-cell tumors. Most of the other drugs (for example, Tirzepatide™, Liraglutide™, and Dulaglutide™) used to treat central obesity and diabetes also share the aforementioned side effects.

Therefore, in view of the above-mentioned side effects associated with the popular drugs used for treating and alleviating metabolic disorders such as central obesity and diabetes, there is felt a particular need for an effective, safe, and appropriate treatment for metabolic disorders in mammals, especially human beings. There is felt a particular need for a safe-to-use herbal composition that alleviates and treats the symptoms associated with central obesity, stress-induced fatigue, and type 2 diabetes, and promotes lean muscle mass and a reduction in body fat, body mass index, and body weight.

The present disclosure is directed to a safe-to-consume and side-effect-free herbal composition for the treatment of metabolic disorders in mammals, especially human beings.

Another objective of the present disclosure is directed to a herbal composition that lowers fat absorption in the human body and improves insulin signaling.

Another objective of the present disclosure is directed to a herbal composition that has been clinically validated for its therapeutic efficacy against metabolic disorders.

Another objective of the present disclosure directed to a herbal composition that produces sustainable weight management and bodily energy management-related results, and is not habit-forming even in the long run.

Another objective of the present disclosure is directed to a herbal composition that does not cause any adverse side effects and provides the desirable therapeutic effect within a reasonable period of time.

Another objective of the present disclosure is directed to a herbal composition that, when consumed according to a prescribed dosage, reduces weight and body fat, improves the body mass index (BMI), and enhances adiponectin levels, lean muscle mass, and energy levels in the human body.

Another objective of the present disclosure is directed to a herbal composition that, when consumed according to the prescribed dosage, reduces the accumulation of adipose tissue in the human body and decreases cholesterol levels, triglyceride levels, and low-density lipoprotein levels in the human body, all the while enhancing high-density lipoprotein levels.

A further objective of the present disclosure is directed to a herbal composition that, when consumed according to the prescribed dosage, reduces the levels of TNF-α (Tumor Necrosis Factor-Alpha) and modulates the levels of interleukin-6 (IL-6) in the human body.

Yet another objective of the present disclosure is directed to a herbal composition that, when consumed according to the prescribed dosage, improves liver functionality and brings about a reduction in the organ weight.

Another objective of the present disclosure is directed to a herbal composition that, when consumed according to the prescribed dosage, improves the body composition and the lipid profile, reduces the body weight, reduces the insulin resistance in the human body, and brings about a significant reduction in stress-induced fatigue.

Another objective of the present disclosure is directed to a herbal composition that is safe for consumption in terms of cardiovascular effects and offers improved tolerability.

Yet another objective of the present disclosure is directed to a herbal composition that effectively activates a multitude of metabolic pathways that, in turn, support and accelerate fat loss and energy recovery.

### SUMMARY

The present disclosure is directed to a herbal composition that treats a plurality of metabolic disorders in mammals, especially human beings. The metabolic disorders treated by the herbal composition include central obesity, stress-induced fatigue, high blood pressure levels, high blood sugar levels, diabetes, and dyslipidemia. The herbal composition of the present disclosure promotes fat loss, improves body mass composition, and enhances energy recovery. The herbal composition of the present disclosure preferably includes the extracts of Dolichos lablab (common name: hyacinth bean, Indian bean), Achyranthes Aspera (prickly chaff flower), and at least one of Cinnamomum zeylanicum (common name: Cinnamon, Ceylon Cinnamon, true Cinnamon) and Cinnamomum Cassia (common name: Chinese cinnamon).

In accordance with the present disclosure, the herbal composition comprises the extract of Dolichos lablab in an amount ranging between 0.1% to 57.38% by weight of the herbal composition. In accordance with the present disclosure, the herbal composition comprises the extract of Achyranthes Aspera in an amount ranging between 0.1% to 32.79% by weight of the herbal composition. Preferably, the herbal composition comprises the extract of at least one of Cinnamomum zeylanicum and Cinnamomum Cassia in an amount ranging between 0.1% to 9.83% by weight of the herbal composition.

In accordance with the present disclosure, the herbal composition contains the extracts of Dolichos lablab, Achyranthes Aspera, Cinnamomum zeylanicum, and Cinnamomum Cassia. In an alternative embodiment, the herbal composition contains at least one of the fractions, active compounds, and phytochemicals derived from the plant parts of the above-identified herbs, viz., Dolichos lablab, Achyranthes Aspera, Cinnamomum zeylanicum, and Cinnamomum Cassia.

In accordance with the present disclosure, the extracts, fractions, active compounds, or phytochemicals are derived from at least one of the leaves, stems, tender stems, tender twigs, aerial parts, whole fruits, fruit rinds, seeds, roots, bark, and hardwood parts of Dolichos lablab, Achyranthes Aspera, Cinnamomum zeylanicum, and Cinnamomum Cassia.

In accordance with the present disclosure, the herbal composition can be formulated as an oral dosage form. The herbal composition can be formulated as a tablet, capsule, soluble powder, chewable tablet, chewable gum, liquefied beverage, or chewable candy.

In accordance with the present disclosure, the herbal composition is administered to a human being on an empty stomach and in the form of a five-hundred milligram (mg) capsule once a day, per every kilogram (kg) bodyweight of a human being, 7.15 milligrams (mg) of the herbal composition is administered, with the dosage of the herbal composition being represented as “7.15 mg/kg bodyweight”. Preferably, the herbal composition, in the form of a capsule, is administered at least thirty minutes before meals.

In accordance with the present disclosure, the herbs, viz., Dolichos lablab, Achyranthes Aspera, and at least one of Cinnamomum zeylanicum and Cinnamomum Cassia, are ground and subsequently extracted using appropriate solvents under a controlled temperature and controlled pH (potential of Hydrogen) value. For instance, the solvent used for preparing the extracts of the above-identified herbs contains water and ethanol in a predetermined ratio and is maintained at a predetermined pH value.

The extracts of Dolichos lablab, Achyranthes Aspera, and at least one of Cinnamomum zeylanicum and Cinnamomum Cassia, thus obtained, are concentrated using the process of solvent evaporation, the process of chromatography, or a process that includes both ultrafiltration and nanofiltration. The extracts of the above-identified herbs are concentrated until a desired assay of active compounds is obtained from each of the extracts of Dolichos lablab, Achyranthes Aspera, and Cinnamomum zeylanicum (or Cinnamomum Cassia). Preferably, the herbal composition comprises active compounds extracted and quantified from the extracts of Dolichos lablab, Achyranthes Aspera, and Cinnamomum zeylanicum (or Cinnamomum Cassia). Preferably, the active compounds extracted and quantified include saponins, proanthocyanidins (PA), tannins, and polyphenols.

The proanthocyanidins are extracted and quantified from the extracts of Dolichos lablab, Achyranthes Aspera, and Cinnamomum zeylanicum (or Cinnamomum Cassia) by the process of UV-Vis spectrophotometry. Likewise, tannins and polyphenols are also extracted and quantified from the extracts of Dolichos lablab, Achyranthes Aspera, and Cinnamomum zeylanicum (or Cinnamomum Cassia) by the process of UV-Vis spectrophotometry. Furthermore, saponins are extracted and quantified from the extracts of Dolichos lablab, Achyranthes Aspera, and Cinnamomum zeylanicum (or Cinnamomum Cassia) by the process of gravimetry.

The herbal composition envisaged by the present disclosure contains proanthocyanidins in a concentration ranging between 0.1% and 20% by weight of the herbal composition, with the proanthocyanidins exhibiting a purity of at least 2%. Likewise, the herbal composition contains saponins in a concentration ranging between 0.1% and 50% by weight of the herbal composition, with the Saponins exhibiting a purity of at least 15%. Likewise, the herbal composition contains polyphenols in a concentration ranging between 0.1% and 10% by weight of the herbal composition, with the Polyphenols exhibiting a purity of at least 2%. Further, the herbal composition also contains tannins in a concentration ranging between 0.1% and 20% by weight of the herbal composition, with the tannins exhibiting a purity of at least 3%.

The herbal composition brings about a significant enhancement in the adiponectin levels, thereby alleviating the symptoms associated with insulin resistance (viz., the onset of type 2 diabetes), improving cardiovascular health, and promoting anti-inflammatory and anti-atherogenic effects. Furthermore, the herbal composition also causes a reasonable increase in the high-density lipoprotein (HDL) levels, with the HDL protecting the human body against cardiovascular diseases by removing excess cholesterol arteries and causing the kidneys to dispose the excess cholesterol, thereby lowering the risk of heart attack and stroke. In accordance with the present disclosure, the herbal composition also brings about a reduction in the accumulation of adipose tissue (in the human body), total cholesterol levels, triglyceride levels, and low-density lipoprotein (LDL) levels. Typically, the adipose tissue, which is also referred to as body fat, is formed when excess nutrients are stored in the form of triglycerides in the human body. The excess fat stored in the human body in the form of adipose tissue causes central obesity, where existing fat cells enlarge and new fat cells take form. Furthermore, central obesity leads to systemic inflammation, insulin resistance, and enhances the risk of metabolic diseases such as type 2 diabetes and cardiovascular diseases. The herbal composition, by preventing the accumulation of adipose tissue, brings down the triglyceride levels significantly, treats and alleviates central obesity, and alleviates the symptoms typically associated with central obesity, for example, insulin resistance and the consequential onset of type 2 diabetes, and significantly reduces the risk of heart attack and stroke.

Further, the herbal composition envisaged by the present disclosure, when administered as per the prescribed dosage of five hundred milligrams per day, also brings down the total cholesterol levels and low-density lipoprotein (LDL) levels in the human body, thereby helping prevent cardiovascular diseases such as a heart attack and stroke, and the buildup of plaque (atherosclerosis) in the arteries. Further, the herbal composition, by lowering the LDL levels, also helps halt and reverse the progression of existing plaque.

Further, the herbal composition envisaged by the present disclosure, when administered as per the prescribed dosage of five-hundred milligrams per day, exhibits enhanced activity at the glucagon-like peptide-1 (GLP-1) receptor, which stimulates the pancreas to secrete insulin and suppress glucagon (a hormone that raises blood sugar), thereby slowing down the process of digestion and slowing the appetite, thereby promoting feelings of fullness. The herbal composition functions like a GLP-1 agonist and mimics the GLP-1 hormone to treat and alleviate central obesity and type 2 diabetes by regulating the body metabolism and causing weight loss. Furthermore, the herbal composition envisaged by the present disclosure partially and selectively (viz., in a controlled manner) inhibits the Monoacylglycerol Acyltransferase 2 (MGAT2) enzyme, thereby controlling the amount of dietary fat absorbed by the human body, and further influencing the efficiency of energy metabolism and the regulation of fat intake. By inhibiting the MGAT2 enzyme in a controlled manner, the herbal composition controls the resynthesis of triacylglycerol (TAG) from monoacylglycerols and fatty acids, thereby controlling the rate at which dietary fat is absorbed into the human body and influencing the lipid metabolism of the human body.

### BRIEF DESCRIPTION OF THE ACCOMPANYING DRAWINGS

FIG. 1 is a clustered column graph illustrating the changes observed in the total cholesterol levels of the human subjects who participated in a clinical study conducted to establish the efficacy of the herbal composition envisaged by the present disclosure.

FIG. 2 is a clustered column graph illustrating the changes observed in the triglyceride levels of the human subjects who participated in the clinical study.

FIG. 3 is a clustered column graph illustrating the changes observed in LDL cholesterol levels of the human subjects who participated in the clinical study.

FIG. 4 is a clustered column graph illustrating the changes observed in HDL cholesterol levels of the human subjects who participated in the clinical study.

FIG. 5 is a clustered column graph illustrating the changes observed in adiponectin levels of the human subjects who participated in the clinical study.

FIG. 6 is a clustered column graph illustrating the changes observed in the body fat percentage of the human subjects who participated in the clinical study.

FIG. 7 is a clustered column graph illustrating the changes observed in the lean body mass of the human subjects who participated in the clinical study.

FIG. 8 is a clustered column graph illustrating the changes observed in the waist circumference of the human subjects who participated in the clinical study.

FIG. 9 is a clustered column graph illustrating the changes observed in the hip circumference of the human subjects who participated in the clinical study.

FIG. 10 is a clustered column graph illustrating the changes observed in the waist-to-hip ratio (WHR) of the human subjects who participated in the clinical study.

FIG. 11 is a clustered column graph illustrating the changes observed in the general fatigue levels of the human subjects who participated in the clinical study.

FIG. 12 is a clustered column graph illustrating the changes observed in the physical fatigue levels of the human subjects who participated in the clinical study.

FIG. 13 is a clustered column graph illustrating the changes observed in the reduced activity score corresponding to the human subjects who participated in the clinical study.

FIG. 14 is a clustered column graph illustrating the changes observed in the reduced motivation score corresponding to the human subjects who participated in the clinical study.

FIG. 15 is a clustered column graph illustrating the changes observed in the cognitive fatigue levels of the human subjects who participated in the clinical study.

FIG. 16 is a clustered column graph illustrating the changes observed in the total fatigue levels of the human subjects who participated in the clinical study.

FIG. 17 is a clustered column graph illustrating the changes observed in the body mass index (BMI) of the human subjects who participated in the clinical study.

FIG. 18 is a clustered column graph illustrating the changes observed in the body weight of the human subjects who participated in the clinical study.

### DETAILED DESCRIPTION

The present disclosure is directed to a herbal composition for treating and alleviating a multitude of metabolic disorders in mammals, especially human beings. Typically, metabolic disorders are conditions that disrupt the process by which the human body converts food into energy, which, in turn, is one of the basic building blocks for creating and sustaining human body cells. Metabolic disorders are typically characterized by abnormalities in carbohydrate, lipid, or protein metabolism, leading to imbalances in glucose levels, cholesterol levels, and hormone regulation. Typically, metabolic disorders cause a dysregulation of normal biochemical processes associated with energy production, storage, and utilization, and therefore affect the metabolic pathways that govern carbohydrate, lipid, and protein metabolism, thus leading to impaired glucose homeostasis, dyslipidaemia, and abnormal adipose tissue accumulation.

Common examples of metabolic disorders in human beings include abdominal obesity (also referred to as central obesity or visceral obesity), type 2 diabetes, dyslipidaemia, high blood sugar levels (hyperglycaemia), and high blood pressure levels (hypertension), inter alia. Typically, all of the aforementioned metabolic disorders are closely linked with insulin resistance and chronic inflammation. Typically, insulin resistance disrupts cellular glucose uptake and alters lipid synthesis and oxidation. Insulin resistance is often accompanied by mitochondrial dysfunction and low-grade chronic inflammation, which often exacerbate the metabolic imbalance in the human body. Further, metabolic disorders are often caused by wrong lifestyle influences and choices, and if left untreated and unmanaged, increase the risk of cardiovascular diseases (including a heart attack, stroke, and cardiac arrest), systemic liver dysfunction, insulin-dependent diabetes progression, and chronic hormonal imbalance. Clinically, the aforementioned metabolic disorders are influenced mainly by stress (both physical and cognitive), sedentary lifestyle and behaviour, which, in turn, collectively alter endocrine signalling and energy metabolism in the human body.

The herbal composition disclosed contains active compounds (for example, polyphenols, flavonoids, tannins, proanthocyanidins, and saponins), derived from medicinal plants, which play a significant role in modulating key biochemical and metabolic pathways associated with metabolic dysfunction. The active compounds present in the herbal extracts that constitute the herbal composition act on a variety of molecular targets in the human body, including adenosine monophosphate-activated protein kinase (AMPK), peroxisome proliferator-activated receptors (PPARs), and nuclear factor kappa B (NF-κB) to restore metabolic balance and to attenuate inflammatory signalling in the human body. Furthermore, the active compounds contained in the herbal composition exert gut-microbiota-modulating effects and thus positively influence short-chain fatty acid (SCFA) production and bile metabolism, which, in turn, regulate systemic energy homeostasis. Collectively, the active compounds present in the herbal extracts that constitute the herbal composition contribute to improved glycaemic control, lipid regulation, and reduction of adipose inflammation, thereby offering a multi-targeted and effective yet safer therapeutic alternative to synthetic pharmacotherapies in managing metabolic disorders in human beings.

In accordance with the present disclosure, the active compounds, such as polyphenols, flavonoids, and saponins, present in the herbal composition exhibit antioxidant and insulin-sensitizing effects. These active compounds enhance glucose uptake (i.e., absorption of glucose from the blood by body cells), improve mitochondrial function, and reduce reactive oxygen species (ROS).

The herbal composition in the present disclosure comprises the extracts of Dolichos lablab (common name: hyacinth bean, Indian bean), Achyranthes aspera (common name: prickly chaff flower), and Cinnamomum zeylanicum (common name: Cinnamon, Ceylon Cinnamon, true Cinnamon). In accordance with an alternative embodiment of the present disclosure, the herbal composition comprises the extracts of Dolichos lablab, Achyranthes aspera, and the extract of Cinnamomum Cassia (common name: Chinese cinnamon), instead of the extract of Cinnamomum zeylanicum. It is well within the purview of the present disclosure to use different plant parts of the herbs identified hitherto to synthesize the herbal composition of the present disclosure, with the herbs and the plant parts thereof being selected based on their polymeric forms, molecular weights, and the quantum of extractable bioactive compounds contained therein.

In accordance with the present disclosure, the plant parts (especially the seeds and pods) of Dolichos lablab are rich in phytochemicals such as flavonoids, phenolic acids, protease inhibitors, lectins, and saponins. These phytochemicals contribute to antioxidant, anti-obesity, and antihyperglycemic activities. In accordance with the present disclosure, flavonoids present in Dolichos lablab enhance mitochondrial β-oxidation, suppress lipid peroxidation, and improve hepatic antioxidant enzyme activities. Further, the extracts of Dolichos lablab reduce adipocyte differentiation and hepatic triglyceride accumulation, while improving insulin sensitivity. Further, the extracts of Dolichos lablab also provide dietary proteins and resistant starches that, in turn, modulate gut microbiota composition and postprandial glucose metabolism.

In accordance with the present disclosure, the extracts of Achyranthes aspera contain saponins, alkaloids, flavonoids, triterpenoids, and polysaccharides that exhibit hypolipidemic, anti-hyperglycaemic, and anti-inflammatory effects. In accordance with the present disclosure, saponins contained in the extracts of Achyranthes aspera inhibit intestinal lipid absorption, enhance lipoprotein lipase activity, and modulate proliferator-activated receptor-γ (PPAR-γ) expression. Further, the extracts of Achyranthes aspera reduce serum glucose, low-density lipoprotein (LDL) cholesterol, and hepatic fat accumulation, thereby helping manage central obesity and dyslipidaemia in human beings. Furthermore, the extracts of Achyranthes aspera exhibit diuretic and hepatoprotective activities and thus support detoxification and metabolic balance in the human body.

In accordance with the present disclosure, the extracts (especially the bark and leaves) of Cinnamomum zeylanicum contain bioactive phytochemicals such as cinnamaldehyde, eugenol, coumarin, cinnamic acid, and proanthocyanidins (PA). These phytochemicals exhibit strong antioxidant, anti-inflammatory, and insulin-sensitizing properties. In accordance with the present disclosure, cinnamaldehyde contained in the extracts of Cinnamomum zeylanicum activates adenosine monophosphate-activated protein kinase (AMPK) and enhances the insulin receptor tyrosine phosphorylation process (mechanism) and promotes glucose uptake in adipose tissues and muscle tissue, thereby contributing to the management of blood sugar levels. The extracts of Cinnamomum zeylanicum improve glycaemic control, reduce plasma triglycerides and cholesterol, and suppress hepatic lipogenesis. The extracts of Cinnamomum zeylanicum also exhibit antimicrobial effects and microbiota-modulating effects, thereby improving metabolic homeostasis by reducing intestinal inflammation and endotoxin load.

In accordance with the present disclosure, the extracts (especially the bark and twigs) of Cinnamomum cassia contain bioactive compounds such as phenylpropanoids, polyphenols, terpenoids, and aromatic and bioactive oils. In accordance with the present disclosure, the phenylpropanoids contained in the extracts of Cinnamomum cassia enhance insulin receptor (IR) phosphorylation and glucose transporter (GLUT 4) translocation, thereby improving cellular glucose uptake and insulin sensitivity. Further, the extracts of Cinnamomum cassia also activate the adenosine monophosphate-activated protein kinase (AMPK), thereby promoting fatty acid oxidation and reducing hepatic gluconeogenesis. Further, the extracts of Cinnamomum cassia also upregulate proliferator-activated receptor-γ (PPAR-γ) and adiponectin levels, thereby improving lipid metabolism and reducing visceral adiposity in the human body by promoting anti-inflammatory and anti-proliferative effects in the human body. Further, the extracts of Cinnamomum cassia also neutralize excess reactive oxygen species (ROS), suppress the Nuclear Factor kappa-light-chain-enhancer of activated B cells (NF-κB), a protein complex that controls the expression of genes involved in immunity, inflammation, and cell growth, and improve tumour necrosis factor-alpha (TNF-α) signalling, thereby reducing stress (especially oxidative stress) and chronic inflammation. Furthermore, the extracts of Cinnamomum cassia also influence the carbohydrate-generating enzymes and thereby lower postprandial glucose spikes. In accordance with the present disclosure, the extracts of Cinnamomum cassia reduce fasting glucose by HbA1c by promoting insulin sensitization. Further, the extracts of Cinnamomum cassia improve the lipid profile by lowering total cholesterol, triglycerides, and low-density lipoprotein (LDL) and by enhancing high-density lipoprotein (HDL). Further, the extracts of Cinnamomum cassia help manage obesity by modulating adipocyte differentiation and fat accumulation. Further, the extracts of Cinnamomum cassia also help reduce hypertension.

In accordance with the present disclosure, the herbal composition is formulated as about a five-hundred milligrams (mg) capsules. The dosage for human beings is about one five-hundred capsule per day, preferably half an hour before meals. Further, in accordance with the present disclosure, the dosage of the herbal composition is about 7.15 milligrams (mg) per every kilogram (kg) bodyweight of the human being administered the herbal composition, i.e., 7.15 mg/kg bodyweight.

The herbal composition envisaged by the present disclosure, when consumed as per the aforementioned prescribed dosage, reduces bodily inflammation and also brings about a decrease in body mass. The herbal composition envisaged by the present disclosure also promotes weight loss, improved energy recovery and retention, lower blood sugar levels, improved blood pressure levels, and improved lipid profile. Furthermore, the herbal composition, when consumed as per the aforementioned prescribed dosage, also triggers a decrease in the cholesterol levels, triglyceride levels, and low-density lipoprotein levels, all three indicative of improved heart health and reduced risk of cardiovascular diseases. The herbal composition also brings about an elevation in the high-density lipoprotein (HDL) levels, with higher HDL levels indicating reduced plaque formation in the blood-carrying arteries and improved heart health.

The herbal composition envisaged by the present disclosure also brings about a reduction in the levels of tumor necrosis factor-alpha (TNF-α) and prevents the over-production of tumor necrosis factor-alpha, a crucial cytokine that, when over-produced, promotes inflammation and acts as a contributory factor to major autoimmune and inflammatory diseases such as rheumatoid arthritis and Crohn's disease. Further, the herbal composition, when consumed as per the aforementioned prescribed dosage, also enhances the adiponectin levels, with the enhanced adiponectin levels alleviating central obesity, promoting improved metabolic health, and reducing the risk of cardiovascular diseases.

Further, the herbal composition, when consumed as per the aforementioned prescribed dosage, also modulates the interleukin-6 (IL-6) levels, which, in turn, act as an inflammatory marker, with higher levels of interleukin-6 indicating inflammation and a possible infection. In accordance with the present disclosure, the herbal composition, by modulating the levels of interleukin-6 (IL-6), alleviates the symptoms associated with cardiovascular diseases, rheumatoid arthritis, lupus, stroke, and type 2 diabetes. Further, the herbal composition, when consumed as per the aforementioned prescribed dosage, also brings about a decrease in the levels of biomarkers, viz., bilirubin, albumin time, prothrombin time, Aspartate Aminotransferase (AST), Alanine Aminotransferase (ALT), Alanine Transaminase, Aspartate Transaminase, Alkaline Phosphatase, and Gamma-Glutamyl Transferase, typically used for liver function tests (LFT) and for indicating abnormalities in liver function, and specifically, liver damage.

Further, the herbal composition, when consumed as per the aforementioned prescribed dosage, brings about a significant decrease in the body weight of human beings. Further, the herbal composition also brings about a significant decrease in the organ weight, especially the liver weight. Further, the herbal composition also brings about a significant decrease in the epididymal fat, which serves as a fat storage (fat depot) in human beings. Further, the herbal composition also brings about a significant decrease in the subcutaneous fat, which is located just below the skin (epidermal layer). The herbal composition, when consumed as per the aforementioned prescribed dosage, modifies a multitude of metabolic pathways that support fat loss and energy recovery. Specifically, the herbal composition inhibits the Monoacylglycerol Acyltransferase 2 (MGAT2) enzyme, which, in turn, synthesizes triglycerides, thereby reducing triglyceride levels in the human body and lowering fat absorption, and thereby alleviating and treating the symptoms associated with central obesity and type 2 diabetes. Furthermore, the herbal composition acts as a glucagon-like peptide-1 (GLP-1) receptor agonist and mimics the actions of GLP-1, a natural gut hormone, thereby lowering blood sugar levels by enhancing insulin secretion and reducing glucagon release. Further, by acting as a GLP-1 receptor agonist, the herbal composition also controls the appetite and slows down the process of digestion, thereby promoting weight loss and improvements in lipid profile and blood pressure levels.

In accordance with the present disclosure, the herbal composition comprises the extract of Dolichos lablab in an amount ranging between about 0.1% to about 57.38% by weight of the herbal composition. Further, the herbal composition comprises the extract of Achyranthes aspera in an amount ranging between about 0.1% to about 32.79% by weight of the herbal composition. Furthermore, the herbal composition comprises the extract of Cinnamomum zeylanicum in an amount ranging between about 0.1% to about 9.83% by weight of the herbal composition. In an alternative embodiment, along with the extracts of Dolichos lablab and Achyranthes aspera, the herbal composition comprises the extract of Cinnamomum cassia in an amount ranging between about 0.1% to about 9.83% by weight of the herbal composition, instead of the extract of Cinnamomum zeylanicum.

A polyherbal composition such as the herbal composition in the present disclosure, Cinnamomum cassia serves as a potent insulin sensitizer and carbohydrate regulator, complementing the lipid-modulating and antioxidant actions of Dolichos lablab and Achyranthes aspera. In accordance with the present disclosure, the extract of Cinnamomum zeylanicum contains larger proportions of Cinnamaldehyde (which enhances insulin receptor phosphorylation and glucose transporter translocation, and thereby improves cellular glucose uptake and insulin sensitivity) in comparison to the extracts of Cinnamomum cassia of the same weight. In addition, the extract of Cinnamomum cassia contains larger proportions of coumarin, an anti-inflammatory organic chemical compound, in comparison to the extracts of Cinnamomum zeylanicum of the same weight. In addition, while the extracts of Cinnamomum zeylanicum exhibit antioxidant and anti-inflammatory properties, the extracts of Cinnamomum cassia exhibit antidiabetic and insulin mimetic properties. Therefore, in accordance with the present disclosure, the extracts of one of Cinnamomum cassia and Cinnamomum zeylanicum are used along with the extracts of Dolichos lablab and Achyranthes aspera to formulate the herbal composition.

In accordance with the present disclosure, the plant parts of Dolichos lablab, viz., a combination involving any of the leaves, stems, tender stems, tender twigs, aerial parts, whole fruits, fruit rinds, seeds, roots, bark, and hardwood, are ground and subsequently subjected to a hydroalcoholic extraction process. Seeds, pods, and leaves of Dolichos lablab are preferred for preparing the Dolichos lablab extract.

In accordance with the present disclosure, the hydroalcoholic extraction process for formulating the extract of Dolichos lablab involves the usage of a mixed solvent (first solvent mixture) that contains ethanol and water in a controlled ratio. Preferably, the first solvent mixture contains about 60% to about 65% ethanol by weight and about 35% to about 40% water by weight. The hydroalcoholic extraction process for preparing the extract of Dolichos lablab is typically performed at a temperature ranging between about 60 and about 65 degrees Celsius, with the pH value of the first solvent mixture being maintained between about 4 and about 7. In accordance with the present disclosure, the ratio of the first solvent mixture to the plant parts of Dolichos lablab is maintained at about 1:10, i.e., one gram of plant part of Dolichos lablab and ten milliliters of the first solvent mixture.

In accordance with the present disclosure, ethanol (60%-65%) extracts moderately polar and non-polar active compounds such as flavonoids, isoflavones, phytosterols, fatty acids, lipids, alkaloids, and non-polar saponins. In accordance with the present disclosure, water (35%-40%) helps dissolve highly polar active compounds such as phenolic acids, glycosides, polysaccharides, proteins, peptides, amino acids, and polar saponins. Typically, the first solvent mixture provides a balanced polarity range during the preparation of the extract of Dolichos lablab by ensuring maximum recovery of Dolichos lablab's active compounds.

In accordance with the present disclosure, powdered Dolichos lablab plant parts (preferably the seeds, pods, and leaves) are added to the first solvent mixture that contains ethanol (60%-65%) and water (35%-40%). Subsequently, the first solvent mixture containing the powdered Dolichos lablab plant parts is maintained at a temperature ranging between 60 and 65 degrees Celsius and at a pH value ranging between 4 and 7, under stirring, for a predetermined time period, preferably, 2-4 hours. The first solvent mixture penetrates the plant matrix (viz., the plant parts of Dolichos lablab) dissolving both hydrophilic (water-soluble) constituents (viz., phenolic acids, proteins, polysaccharides, peptides, saponins, and amino acids) and hydrophobic (ethanol-soluble) constituents (viz., flavonoids, isoflavones, phytosterols, fatty acids and lipids, and the like) of the Dolichos lablab plant parts. Subsequently, the first solvent mixture is filtered to remove the residue and is concentrated under reduced pressure to remove ethanol and yield a hydroalcoholic extract of Dolichos lablab. Preferably, the extract of Dolichos lablab thus formulated contains active compounds, including flavonoids, saponins, phenolic acids, isoflavones, proteins, and peptides.

In accordance with the present disclosure, the plant parts of Achyranthes aspera, viz., a combination involving any of the leaves, stems, tender stems, tender twigs, aerial parts, whole fruits, fruit rinds, seeds, roots, bark, and hardwood, are ground and subsequently subjected to the hydroalcoholic extraction process. The aerial parts of Achyranthes aspera are preferred for formulating the extract of Achyranthes aspera. The hydroalcoholic extraction process for formulating the extract of Achyranthes aspera involves the usage of a mixed solvent (second solvent mixture) with a medium polarity that contains ethanol and water in a controlled ratio. Preferably, the second solvent mixture contains about 50% to about 55% ethanol by weight and about 45% to about 50% water by weight. The hydroalcoholic extraction process for preparing the extract of Achyranthes aspera is typically performed at a temperature ranging between about 55 and about 60 degrees Celsius, with the pH value of the second solvent mixture being maintained between about 4 and about 7. In accordance with the present disclosure, the ratio of the second solvent mixture to the plant parts of Achyranthes aspera is in the range between about 1:8 and about 1:10.

In accordance with the present disclosure, ethanol (50%-55%) extracts moderately polar and non-polar active compounds such as flavonoids, phenolics, phytosterols, triterpenoids, fixed oils, lipids, and alkaloids. In accordance with the present disclosure, water (45%-50%) helps dissolve highly polar active compounds such as saponins, phenolic acids, polysaccharides, amino acids, peptides, and alkaloid salts. Typically, the second solvent mixture provides a midrange polarity during the preparation of the extract of Achyranthes aspera by ensuring maximum recovery of Achyranthes aspera's bioactive compounds without causing a degradation thereof.

In accordance with the present disclosure, powdered Achyranthes aspera plant parts are added to the second solvent mixture that contains ethanol (50%-55%) and water (45%-50%). Subsequently, the second solvent mixture containing the powdered Achyranthes aspera plant parts is maintained at a temperature ranging between 55 and 60 degrees Celsius and at a pH value ranging between 4 and 7, under stirring or reflux, for a predetermined time period, preferably, 2-4 hours. The second solvent mixture penetrates the plant matrix (plant parts of Achyranthes aspera), dissolving both hydrophilic (water-soluble) constituents (viz., saponins, phenolic acids, polysaccharides, amino acids, peptides, and alkaloid salts) and hydrophobic (ethanol-soluble) constituents (viz., flavonoids, phenolics, phytosterols, triterpenoids, fixed oils, lipids, and alkaloids) of the Achyranthes aspera plant parts. Subsequently, the second solvent mixture is filtered to remove the residue and is concentrated under reduced pressure to remove ethanol and yield a hydroalcoholic extract of Achyranthes aspera. Preferably, the extract of Achyranthes aspera thus formulated contains active compounds, including polar saponins, flavonoids, and alkaloids.

In accordance with the present disclosure, the plant parts of Cinnamomum zeylanicum, viz., a combination involving any of the leaves, stems, tender stems, tender twigs, aerial parts, whole fruits, fruit rinds, seeds, roots, bark, and hardwood, are ground and subsequently subjected to the hydroalcoholic extraction process. The bark and leaves of Cinnamomum zeylanicum are preferred for formulating the extract of Cinnamomum zeylanicum. The hydroalcoholic extraction process for formulating the extract of Cinnamomum zeylanicum involves the usage of a mixed solvent (third solvent mixture) with a medium polarity that contains ethanol and water in a controlled ratio. The third solvent mixture is designed to extract a wide polarity range of bioactive compounds. The third solvent mixture contains about 60% to about 65% ethanol by weight and about 35% to about 40% water by weight. The hydroalcoholic extraction process for preparing the extract of Cinnamomum zeylanicum is typically performed at a temperature ranging between about 55 and about 60 degrees Celsius, with the pH value of the third solvent mixture being maintained between about 3 and about 5. In accordance with the present disclosure, the ratio of the third solvent mixture to the plant parts of Cinnamomum zeylanicum is maintained at 1:8, i.e., one gram of plant part of Cinnamomum zeylanicum and ten milliliters of the third solvent mixture.

In accordance with the present disclosure, ethanol (60%-65%) extracts moderately polar and non-polar active compounds such as aromatic aldehydes, coumarins, flavonoids, essential oils, and triterpenoids. In accordance with the present disclosure, water (35%-40%) helps dissolve highly polar active compounds such as phenolic acids, tannins, glycosides, polysaccharides, and water-soluble saponins. Typically, the third solvent mixture extracts volatile oil components (viz., essential oils) from the oil glands of the bark of Cinnamomum zeylanicum without full dehydration. Further, in addition to a high recovery of essential oils, the third solvent solution also facilitates a high recovery of phenolic acids, thereby facilitating balanced antioxidant and insulin-sensitizing effects.

In accordance with the present disclosure, powdered Cinnamomum zeylanicum plant parts are added to the third solvent mixture that contains ethanol (60%-65%) and water (35%-40%). Subsequently, the third solvent mixture containing the powdered Cinnamomum zeylanicum plant parts is maintained at a temperature ranging between about 55 and about 60 degrees Celsius and at a pH value ranging between about 3 and about 5, under stirring or reflux, for a predetermined time period, at about 2-3 hours. The third solvent mixture penetrates the plant matrix (plant parts of Cinnamomum zeylanicum), dissolving both hydrophilic (water-soluble) constituents (viz., phenolic acids, tannins, glycosides, polysaccharides, and water-soluble saponins) and hydrophobic (ethanol-soluble) constituents (viz., aromatic aldehydes, coumarins, flavonoids, essential oils, and triterpenoids) of the Cinnamomum zeylanicum plant parts. Subsequently, the third solvent mixture is filtered to remove the residue and is concentrated under reduced pressure to remove ethanol and yield a hydroalcoholic extract of Cinnamomum zeylanicum. Preferably, the extract of Cinnamomum zeylanicum thus formulated contains active compounds, including tannins, polysaccharides, essential oils, phenolics, and water-soluble saponins.

In accordance with an alternative embodiment of the present disclosure, instead of the plant parts of Cinnamomum zeylanicum, the plant parts of Cinnamomum cassia are used for the formulation of the herbal composition. Even in the case of Cinnamomum cassia, a combination of any of the leaves, stems, tender stems, tender twigs, aerial parts, whole fruits, fruit rinds, seeds, roots, bark, and hardwood is used for preparing the extract of Cinnamomum cassia. The bark and twigs of Cinnamomum zeylanicum are preferred for formulating the extract of Cinnamomum cassia. In accordance with the present disclosure, the plant parts of Cinnamomum cassia are ground and subsequently subjected to the hydroalcoholic extraction process. The hydroalcoholic extraction process for formulating the extract of Cinnamomum cassia involves the use of the third solvent mixture that, as explained above, contains about 60% to about 65% ethanol by weight and about 35% to about 40% water by weight. The hydroalcoholic extraction process for preparing the extract of Cinnamomum cassia is also performed at a temperature ranging between about 55 and about 60 degrees Celsius, with the pH value of the third solvent mixture being maintained between about 3 and about 5. In accordance with the present disclosure, the ratio of the third solvent mixture to the plant parts of Cinnamomum cassia is in the range between about 1:8 and about 1:10, i.e., one gram of the plant parts of Cinnamomum cassia and eight to ten milliliters of the third solvent mixture.

The third solvent mixture, containing about 60% to about 65% ethanol and about 35% to about 40% water, is suitable for extracting the essential oils and cinnamaldehyde from the bark of Cinnamomum cassia. In accordance with the present disclosure, ethanol (60%-65%) extracts moderately polar and non-polar active compounds such as essential oils (viz., cinnamyl acetate, linalool, eugenol), cinnamaldehyde, coumarins, and flavonoids. In accordance with the present disclosure, water (35%-40%) helps dissolve highly polar active compounds such as phenolic acids, tannins, glycosides, polysaccharides, and water-soluble flavonoids.

Typically, the third solvent mixture extracts key aromatic active compounds such as cinnamaldehyde and coumarin that are sparingly soluble in water, while water retains polar antioxidants such as phenolics and tannins. In accordance with the present disclosure, powdered Cinnamomum cassia plant parts are added to the third solvent mixture that contains ethanol (60%-65%) and water (35%-40%). Subsequently, the third solvent mixture containing the powdered Cinnamomum cassia plant parts is maintained at a temperature ranging between about 55 and about 60 degrees Celsius and at a pH value ranging between about 3 and about 5, under stirring or reflux, for a predetermined time period, about 2-3 hours. The third solvent mixture penetrates the plant matrix (plant parts of Cinnamomum cassia), dissolving both hydrophilic (water-soluble) constituents (viz., phenolic acids, tannins, glycosides, polysaccharides, and water-soluble flavonoids) and hydrophobic (ethanol-soluble) constituents (viz., essential oils, cinnamaldehyde, coumarins, and flavonoids) of the Cinnamomum cassia plant parts. Subsequently, the third solvent mixture is filtered to remove the residue and is concentrated under reduced pressure to remove ethanol and yield a hydroalcoholic extract of Cinnamomum cassia. Preferably, the extract of Cinnamomum cassia thus formulated contains active compounds, including tannins, cinnamaldehyde, glycosides, coumarins, essential oils, and phenolics.

In accordance with the present disclosure, instead of ethanol, the first solvent mixture, the second solvent mixture, and the third solvent mixture comprise at least one of methanol, n-propanol, and isopropyl alcohol, in an amount ranging between about 60% to about 65% by weight of the first solvent mixture, about 50% to about 55% by weight of the second solvent mixture, and about 60% to about 65% by weight of the third solvent mixture, respectively.

Alternatively, the first solvent mixture, the second solvent mixture, and the third solvent mixture comprise ketones, including at least one of acetone and methyl isobutyl ketone, in an amount ranging between about 60% to about 65% by weight of the first solvent mixture, about 50% to about 55% by weight of the second solvent mixture, and about 60% to about 65% by weight of the third solvent mixture, respectively.

Alternatively, the first solvent mixture, the second solvent mixture, and the third solvent mixture comprise C1-C7 hydrocarbons, including at least one of methane, ethane, propane, butane, pentane, hexane, and heptane, in an amount ranging between about 60% to about 65% by weight of the first solvent mixture, about 50% to about 55% by weight of the second solvent mixture, and about 60% to about 65% by weight of the third solvent mixture, respectively.

Alternatively, the first solvent mixture, the second solvent mixture, and the third solvent mixture comprise chlorinated hydrocarbons, including at least one of methylene dichloride and chloroform, in an amount ranging between about 60% to about 65% by weight of the first solvent mixture, about 50% to about 55% by weight of the second solvent mixture, and about 60% to 65% by weight of the third solvent mixture, respectively.

Alternatively, the first solvent mixture, the second solvent mixture, and the third solvent mixture comprise esters (for example, ethyl acetate), including at least one of methylene dichloride and chloroform, in an amount ranging between about 60% to about 65% by weight of the first solvent mixture, about 50% to about 55% by weight of the second solvent mixture, and about 60% to about 65% by weight of the third solvent mixture, respectively.

In accordance with the present disclosure, the extracts of Dolichos lablab, the extracts of Achyranthes aspera, and the extracts of Cinnamomum zeylanicum are mixed to formulate the herbal composition such that the herbal composition comprises the extract of Dolichos lablab in an amount ranging between about 0.1% to about 57.38% by weight of the herbal composition, the extract of Achyranthes Aspera in an amount ranging between about 0.1% to about 32.79% by weight of the herbal composition, and the extract of Cinnamomum zeylanicum in an amount ranging between about 0.1% to about 9.83% by weight of the herbal composition.

In an alternative embodiment of the present disclosure, as discussed hitherto, the herbal composition comprises the extract of Dolichos lablab in an amount ranging between about 0.1% to about 57.38% by weight of the herbal composition, the extract of Achyranthes aspera in an amount ranging between about 0.1% to about 32.79% by weight of the herbal composition, and the extract of Cinnamomum cassia in an amount ranging between about 0.1% to about 9.83% by weight of the herbal composition.

In accordance with the present disclosure, the herbal composition comprises at least one additional component selected from the group consisting of pharmaceutically acceptable active ingredients, vitamins, minerals, excipients, carriers, and diluents. Preferably, the at least one additional component is present in the herbal composition in an amount ranging between about 1% to about 10% by weight of the herbal composition.

In accordance with the present disclosure, the herbal composition includes carriers and diluents that deliver and/or hold the active compounds (viz., proanthocyanidins, tannins, saponins, and polyphenols) contained in the herbal composition to ensure their proper dispersion, absorption, and release. In accordance with the present disclosure, the carriers a) enhance the solubility of bioactive materials such as saponins and increase their absorption in the digestive tract, b) allow for sustained release of the active herbal components contained in the herbal composition, c) protect sensitive herbal compounds contained in the herbal composition from oxidation, moisture, and heat, and d) deliver active compounds directly to target tissues and thereby improve the efficacy of the herbal composition and reduce the side effects, if any.

In accordance with the present disclosure, diluents are added to the herbal composition to bulk up the formulation for appropriate dosing and improved product handling. In accordance with the present disclosure, the diluents are added to the herbal composition to a) add volume to any small concentrations of active compounds contained within the herbal composition, b) distribute the active compounds evenly throughout the herbal composition, thereby ensuring uniform capsule fill or uniform tablet size, c) absorb moisture, prevent clumping, and increase the shelf-life and stability of the herbal composition, d) prevent sticking or clogging during manufacturing, thereby ensuring smooth production, and e) improve tablet compressibility and mechanical strength (in the event the herbal composition is formulated as a tablet) and prevent breakage during transport and handling.

In accordance with the present disclosure, the carriers and diluents are selected from a group consisting of polyhydric alcohols, sugar alcohols, cellulose-based derivatives, silicates, metallic stearates, organic acids, fatty acid esters, esters of polysorbate, Vitamin B, nicotinamide, calcium pantothenate, amino acids, proteins, and organic metal salts.

In accordance with the present disclosure, the pharmaceutically acceptable active ingredients contained in the herbal composition can include: a) absorption boosters that enhance the bioavailability of herbal compounds contained in the herbal composition by inhibiting drug-metabolizing enzymes in the liver, c) liposomal carriers and phospholipids that improve the delivery of fat-soluble active compounds contained in the herbal composition.

In accordance with the present disclosure, the herbal composition comprises a combination of Vitamins selected from a group consisting of Vitamin D3, Vitamin B6, Vitamin B12, Vitamin E, Vitamin C, and Vitamin A. It is also within the purview of the present disclosure to include all or only some of the above-mentioned vitamins in various possible combinations in the herbal composition. In accordance with the present disclosure, the above-mentioned vitamins, when combined with the herbal composition, support essential cellular functions, promote insulin sensitivity, glucose homeostasis, and fat metabolism, improve blood glucose profile, and lipid profile in human beings who are administered the herbal composition as per the prescribed dose.

In accordance with the present disclosure, the herbal composition comprises at least one mineral selected from the group consisting of Zinc, Magnesium, Selenium, Boron, Chromium, Iron, Calcium, and Copper. It is also within the purview of the present disclosure to include all or only some of the above-mentioned minerals in various possible combinations in the herbal composition. In accordance with the present disclosure, the above-mentioned minerals, when combined with the herbal composition, ensure a) improved absorption of active compounds contained in the herbal composition, b) reduction in oxidative stress and inflammation in the human body, c) insulin regulation in the human body, and d) reduction in stress-related hormone imbalances.

In accordance with the present disclosure, the herbal composition comprises excipients that, even though pharmacologically inactive, significantly influence the herbal composition's efficacy by ensuring the herbal composition's stability, bioavailability, and consistent performance. In accordance with the present disclosure, the excipients a) improve the absorption of the active compounds (viz., proanthocyanidins, saponins, tannins, and polyphenols) contained in the herbal composition by enhancing their solubility and reducing their degradation in the digestive tract, b) prevent the oxidation of active compounds contained in the herbal composition and ensure the stability and the shelf-life of the herbal composition, c) hold the herbal extracts contained in the herbal composition together when the herbal composition is formulated as tablets or capsules, d) facilitate the breakdown of tablets or capsules for faster release of the active compounds contained in the herbal composition, e) protect the active compounds contained in the herbal composition from environmental factors such as moisture and light, f) prevent microbial contamination when the herbal composition is formulated as a liquified beverage or a semi-solid substance, g) aid in the dispersion of herbal compounds contained in the herbal composition, and h) maintain an optimal pH environment for maximum stability and effectiveness of the herbal extracts contained in the herbal composition. In accordance with the present disclosure, the excipients are selected from a group consisting of monosaccharides, disaccharides, polycarbonates, and modified starch.

In accordance with the present disclosure, after the formulation of the herbal composition comprising the extracts of Dolichos lablab, Achyranthes aspera, and at least one of Cinnamomum zeylanicum and Cinnamomum cassia, the first solvent mixture, second solvent mixture, and third solvent mixture, and especially the ethanol contained therein, are removed from the herbal composition under controlled conditions without degrading the active compounds contained in the herbal composition, including proanthocyanidins, saponins, tannins, and polyphenols. In accordance with the present disclosure, the process of solvent evaporation is used to remove the first solvent mixture, the second solvent mixture, and the third solvent mixture, respectively, and the ethanol contained therein.

It is within the purview of the present disclosure to subject the extracts of Dolichos lablab, Achyranthes aspera, and at least one of Cinnamomum cassia and Cinnamomum zeylanicum individually to the process of solvent evaporation, to individually remove the first solvent mixture, second solvent mixture, and third solvent mixture. However, it is also within the purview of the present disclosure to subject the combined extract (that is formed by combining the extracts Dolichos lablab, Achyranthes aspera, and at least one of Cinnamomum cassia and Cinnamomum zeylanicum) to the process of solvent evaporation, to remove the first solvent mixture, the second solvent mixture, and the third solvent mixture together.

In accordance with the present disclosure, after the hydroalcoholic extraction process, the herbal composition (containing the combination of the extracts Dolichos lablab, Achyranthes aspera, and at least one of Cinnamomum cassia and Cinnamomum zeylanicum) is filtered to remove insoluble plant-based residues, and the resultant filtrate is transferred to a rotatory evaporator flask. Subsequently, the rotatory flask is rotated under pressure ranging between 200-400 millibars and at a temperature ranging between 40 and 50 degrees Celsius, while being submerged in a warm water bath. During the rotation of the rotatory flask, the ethanol-water vapors condense in the cooling coil of the rotatory evaporator flask. Preferably, the evaporation process is continued until a thick, viscous extract (containing no ethanol but only the extracts of Dolichos lablab, Achyranthes aspera, and at least one of Cinnamomum cassia and Cinnamomum zeylanicum) remains in the rotatory flask.

In accordance with the present disclosure, the ultrafiltration process and the nanofiltration process are utilized to obtain the desired concentrates/filtrates from the extract obtained from the solvent-evaporation process, i.e., the extract containing Dolichos lablab, Achyranthes aspera, and Cinnamomum cassia or Cinnamomum zeylanicum. In accordance with the present disclosure, the extracts of Dolichos lablab, Achyranthes aspera, and Cinnamomum cassia or Cinnamomum zeylanicum are purified (by ultrafiltration and subsequently, nanofiltration), preferably based on their molecular weights, and by using predetermined ultrafiltration membranes and nanofiltration membranes.

In accordance with the present disclosure, in the ultrafiltration process, membranes with a molecular weight cut-off of 1-100 kDa (Kilodalton; a unit of measurement of molecular weight) are used, whereas in the case of nanofiltration process, membranes with a molecular weight cut-off of 200-1000 Da (Dalton; a unit of measurement of molecular weight) are used. In accordance with the present disclosure, the process of ultrafiltration retains active compounds such as proteins, polysaccharides, large tannins, and aggregated saponins by ultrafiltering the extracts of Dolichos lablab, Achyranthes aspera, and Cinnamomum cassia or Cinnamomum zeylanicum. Further, the process of nanofiltration retains active compounds such as smaller polyphenols, glycosides, proanthocyanidins, flavonoids, and saponins by nano-filtrating the permeates from the ultrafiltration process.

In an embodiment of the present disclosure directed to the implementation of the ultrafiltration process, after the process of solvent evaporation, the extracts of Dolichos lablab, Achyranthes aspera, and at least one of Cinnamomum cassia and Cinnamomum zeylanicum are filtered using coarse filters to remove large-sized debris. Subsequently, in accordance with the present disclosure, the filtered extracts are passed through a Polyether sulfone (PES) membrane, a polyvinylidene fluoride (PVDF) membrane, or a ceramic membrane to filter out (retain) polysaccharides, polyphenols (with higher molecular weight), proteins, large tannins, and aggregated saponins, and similar larger bioactive compounds. For example, a 30-100 kDa ultrafiltration membrane is used to filter out or extract large tannins and aggregated saponins. However, it is within the purview of the present disclosure to use any appropriate ultrafiltration membrane depending upon the polymeric forms and molecular weights of the respective extracts (viz., the extracts of Dolichos lablab, Achyranthes aspera, and at least one of Cinnamomum cassia and Cinnamomum zeylanicum).

In accordance with the present disclosure, during the ultrafiltration process, the temperature is maintained between about 20-35 degrees Celsius to ensure the stability of the bioactive compounds contained within the respective extracts. Furthermore, pressure is maintained between about 2-10 Bar to optimize the flow of the extracts through the ultrafiltration membrane. The permeates from the ultrafiltration process include small-molecule compounds (or compounds with low molecular weight) such as sugars and salts, proanthocyanidins, smaller tannins, polyphenols having a low molecular weight, and non-aggregated saponins, and the retentate (concentrate) from the ultrafiltration process include bioactive compounds such as polysaccharides, polyphenols (with higher molecular weight), proteins, large tannins, and aggregated saponins.

In accordance with the present disclosure, the process of nanofiltration follows the process of ultrafiltration, with the permeates from the ultrafiltration being subjected to the process of nanofiltration. The nanofiltration process retains the smaller polyphenols (having a low molecular weight), proanthocyanidins, glycosides, smaller tannins, and non-aggregated saponins that may not have been retained earlier by the ultrafiltration process. In accordance with the present disclosure, during the process of nanofiltration, the permeates from the ultrafiltration process are filtered yet again using coarse filters to remove solid plant residue. Subsequently, in accordance with the present disclosure, the filtered permeates are passed through micro filters (having a thickness ranging between about 0.2-1.2 μm) to remove fine particulates and bacteria. Subsequently, the micro-filtered permeates are passed through thin-film composite polymeric nano filters or solvent-resistant nano filters to retain smaller polyphenols, smaller tannins, glycosides, proanthocyanidins, flavonoids, and non-aggregated saponins.

An about 200-1000 Da nanofiltration membrane is used to filter out or extract smaller polyphenols, glycosides, proanthocyanidins, flavonoids, smaller tannins, and non-aggregated saponins. However, it is within the purview of the present disclosure to use any appropriate nanofiltration membrane depending upon the polymeric forms and molecular weights of the individual extracts (viz., the extracts of Dolichos lablab, Achyranthes aspera, and at least one of Cinnamomum cassia and Cinnamomum zeylanicum). In accordance with the present disclosure, during the nanofiltration process, the temperature is maintained between 30-40 degrees Celsius to ensure the stability of the bioactive compounds contained within the permeates from the ultrafiltration process. Furthermore, pressure is preferably maintained between about 8-25 Bar to optimize the flow of the ultrafiltered permeates through the nanofiltration membrane. Preferably, the ultrafiltered permeates are passed through the nanofiltration membrane to filter out monovalent salts and small molecules, and to obtain a retentate (concentrate) that is enriched with bioactive compounds such as smaller polyphenols, glycosides, proanthocyanidins, flavonoids, non-aggregated saponins, and smaller tannins. Thus, the herbal composition, after being filtered using the process of ultrafiltration and nanofiltration, is transformed into a purified herbal composition that contains active compounds such as proanthocyanidins, saponins, tannins, and polyphenols in a finer concentration. In accordance with the present disclosure, volatile actives such as cinnamaldehyde and eugenol, that are small enough to permeate through the nanomembranes, are captured separately by distillation. Likewise, very small phenolics that typically permeate through the nanomembranes are retained using adsorption resins.

As discussed hitherto, the herbal composition is formulated by the extract of Dolichos lablab in an amount ranging between about 0.1% to about 57.38% by weight of the herbal composition, the extract of Achyranthes Aspera in an amount ranging between about 0.1% to about 32.79% by weight of the herbal composition, the extract of at least one of Cinnamomum zeylanicum and Cinnamomum Cassia in an amount ranging between about 0.1% to about 9.83% by weight of the herbal composition. Preferably, the herbal composition, filtered by the ultrafiltration and nanofiltration procedures, contains active compounds such as proanthocyanidins, saponins, tannins, and polyphenols. As discussed hitherto, the herbal composition

In accordance with the present disclosure, the herbal composition filtered through the process of ultrafiltration and nanofiltration is subsequently standardized to contain a specified percentage of total proanthocyanidins, saponins, tannins, and polyphenols. Standardization is a quality control specification that sets the minimum acceptable amount of marker compounds or active compounds, or extractable content in the herbal composition. In this case, the active compounds contained in the herbal composition include proanthocyanidins, saponins, tannins, and polyphenols.

In accordance with the present disclosure, the herbal composition is standardized to contain proanthocyanidins in a quantity not less than about 2% by weight of the herbal composition. The proanthocyanidins are polyphenolic compounds that exhibit antioxidant and metabolic regulatory effects, and thereby offer significant therapeutic advantages in treating and alleviating a variety of metabolic disorders plaguing human beings, including central obesity, chronic inflammation, higher blood pressure levels, higher blood sugar levels, and insulin resistance. In accordance with the present disclosure, the herbal composition is standardized to proanthocyanidins (PA) by using a method that involves measuring the DAMC (Dimethylaminocinnamaldehyde) colorimetric assay on a UV-Vis (ultraviolet-visible) spectrometer.

Typically, p-Dimethylaminocinnamaldehyde is used as a reagent and is made to react selectively with flavan-3-ol units in proanthocyanidins to form a colored complex. Typically, the color complex formed after the chemical reaction between the proanthocyanidins and the p-Dimethylaminocinnamaldehyde reagent absorbs light more strongly at about 640 nanometres (with the DMAC-proanthocyanidin complex exhibiting maximum absorbance at 640 nanometres), which lies in the visible region (red-violet part) of the ultraviolet-visible spectrum. Therefore, by using a UV-Vis spectrometer with the wavelength set to 640 nm, the amount of light passing through the herbal composition is measured. Typically, the UV-Vis spectrometer reports an absorbance value, a dimensionless number indicating the amount of light absorbed by the herbal composition. Based on the color intensity exhibited by the chemical reaction between the p-Dimethylaminocinnamaldehyde reagent and the proanthocyanidins contained in the herbal composition, the amount of light absorbed (absorbance value) at 640 nm by the herbal composition is determined. Accordingly, based on the amount of light absorbed, the total concentration of proanthocyanidins in the herbal composition is determined.

The total concentration of proanthocyanidins in the herbal composition is determined by using the p-Dimethylaminocinnamaldehyde (DAMA)-proanthocyanidins complex and the UV-Vis (ultraviolet-visible) spectrometer, with the wavelength set to 640 nm. In accordance with the present disclosure, the total concentration of proanthocyanidins in the herbal composition should not fall below about 2% by weight of the herbal composition. If the total concentration of proanthocyanidins in the herbal composition is determined to be less than about 2% by weight of the herbal composition, then the herbal composition is accordingly re-enriched, for example, by reperforming the nano-filtration of the herbal composition or by performing adsorption resin chromatography.

In accordance with the present disclosure, the herbal composition is standardized to contain saponins in a quantity not less than about 15% by weight of the herbal composition. Saponins are the active compounds that exhibit cholesterol-lowering effects, antioxidant activities, antimicrobial and antifungal properties, and anti-inflammatory, anti-diabetic, and anti-obesity effects, and thereby offer significant therapeutic advantages in treating and alleviating a variety of metabolic disorders plaguing human beings, including central obesity, chronic inflammation, higher blood pressure levels, higher blood sugar levels, insulin sensitivity, and insulin resistance.

In accordance with the present disclosure, the method of gravimetric standardization is used to quantify the total saponin content in the herbal extract. The total saponin content is standardized preferably by treating the herbal composition with aqueous ethanol and thereby dissolving the saponins. The herbal composition is subsequently precipitated using diethyl ether or acetone, thereby causing the saponins to separate from the herbal composition as an insoluble residue. Subsequently, the precipitate is filtered, dried, and weighed to determine the total saponin content in the herbal composition gravimetrically. In accordance with the present disclosure, the total concentration of saponins in the herbal composition should not fall below 15% by weight of the herbal composition. In an event the total concentration of saponins in the herbal composition is determined to be less than 15% by weight of the herbal composition, then the herbal composition is accordingly re-enriched by adjusting or blending individual herbal extracts (viz., the extract of Dolichos lablab and/or the extract of Achyranthes aspera), that contain higher saponin content, with the herbal composition.

In accordance with the present disclosure, the herbal composition is standardized to contain tannins in a quantity not less than about 3% by weight of the herbal composition. The tannins are active polyphenolic compounds that exhibit strong antioxidant activities, antimicrobial properties, anti-inflammatory effects, and antidiarrheal and antiparasitic properties, and thereby offer significant therapeutic advantages in treating and alleviating a variety of metabolic disorders plaguing human beings, including central obesity and chronic inflammation.

In accordance with the present disclosure, the herbal composition is standardized to tannins by using a method that involves measuring the Folin-Ciocalteu colorimetric assay, vanillin-HCl colorimetric assay, or stannous chloride colorimetric assay on a UV-Vis (ultraviolet-visible) spectrometer.

Typically, Folin-Ciocalteu, vanillin-HCl, or stannous chloride is used as a reagent and is made to react with tannins contained in the herbal composition to form a colored complex. Typically, the color complex formed due to the chemical reaction between any of the above-identified reagents and tannins absorbs light more strongly at 540 nanometres, 640 nanometres, and 760 nanometres, depending upon the reagent used (with Folin-Ciocalteu reagent-tannins complex exhibiting maximum absorbance at 760 nanometres, vanillin-HCl reagent-tannins complex exhibiting maximum absorbance at 540 nanometres, and stannous chloride reagent-tannins complex exhibiting maximum absorbance at 640 nanometres). The abovementioned wavelengths lie in the visible region (red-violet part) of the ultraviolet-visible spectrum. Therefore, by using a UV-Vis spectrometer with the wavelength set to 540 nm, 640 nm, or 760 nm (depending upon the reagent used), the amount of light passing through the herbal composition is measured. Typically, the UV-Vis spectrometer reports an absorbance value, a dimensionless number indicating the amount of light absorbed by the herbal composition. Based on the color intensity exhibited by the chemical reaction between the Folin-Ciocalteu, vanillin-HCl, or stannous chloride reagent and tannins contained in the herbal composition, the amount of light absorbed (absorbance value) at 540 nm, 640 nm, or 760 nm nm by the herbal composition is determined. Accordingly, based on the amount of light absorbed, the total concentration of tannins in the herbal composition is determined.

In accordance with the present disclosure, the total concentration of tannins in the herbal composition should not fall below about 3% by weight of the herbal composition. In the event the total concentration of tannins in the herbal composition is determined to be less than about 3% by weight of the herbal composition, then the herbal composition is accordingly re-enriched, for example, by reperforming the ultra-filtration of the herbal composition (to separate high molecular weight tannins from small phenolics) or by performing adsorption resin chromatography.

In accordance with the present disclosure, the herbal composition is standardized to polyphenols (total polyphenol content; PTC) by using a method that involves measuring the Folin-Ciocalteu colorimetric assay on a UV-Vis (ultraviolet-visible) spectrometer. Typically, the polyphenols present in the herbal composition react with the Folin-Ciocalteu reagent under alkaline conditions to form (i.e., to reduce the Folin-Ciocalteu reagent) a blue-coloured complex. The intensity of the blue colour is measured by a UV-Vis spectrometer set to a wavelength of 765 nanometers.

Typically, the color complex formed due to the chemical reaction between the polyphenols and the Folin-Ciocalteu reagent reagent absorbs light more strongly at 765 nanometres (with the Folin-Ciocalteu reagent-polyphenols complex exhibiting maximum absorbance at 765 nanometres), which lies in the visible region (red-violet part) of the ultraviolet-visible spectrum. Therefore, by using the UV-Vis spectrometer set to the wavelength of 765 nm, the amount of light passing through the herbal composition is measured. Typically, the UV-Vis spectrometer reports an absorbance value, a dimensionless number indicating the amount of light absorbed by the herbal composition. Based on the color intensity exhibited by the chemical reaction between the Folin-Ciocalteu reagent and the polyphenols contained in the herbal composition, the amount of light absorbed (absorbance value) at 765 nm by the herbal composition is determined. Accordingly, based on the amount of light absorbed, the total concentration of polyphenols in the herbal composition is determined.

In accordance with the present disclosure, the total concentration of polyphenols in the herbal composition should not fall below 2% by weight of the herbal composition. In an event the total concentration of polyphenols in the herbal composition is determined to be less than about 2% by weight of the herbal composition, then the herbal composition is accordingly re-enriched, for example, by reperforming the ultra-filtration of the herbal composition, Sephadex LH-20 gel filtration (for fractioning low to mid molecular weight phenolics), liquid-liquid partitioning (to partition polyphenols into polar organic fractions), or by performing adsorption resin chromatography.

The herbal composition envisaged by the present disclosure comprises the extract of Dolichos lablab in an amount ranging between about 0.1% to about 57.38% by weight of the herbal composition, the extract of Achyranthes aspera in an amount ranging between about 0.1% to about 32.79% by weight of the herbal composition, the extract of Cinnamomum zeylanicum or the extract of Cinnamomum cassia in an amount ranging between about 0.1% to about 9.83% by weight of the herbal composition.

In accordance with the present disclosure, the herbal composition is standardized to contain proanthocyanidins in a concentration ranging between about 0.1% and about 20% by weight of the herbal composition, saponins in a concentration ranging between about 0.1% and about 50% by weight of the herbal composition, polyphenols in a concentration ranging between about 0.1% and about 10% by weight of the herbal composition, and tannins in a concentration ranging between about 0.1% and about 20% by weight of the herbal composition.

In accordance with the present disclosure, the concentration of the proanthocyanidins in the herbal composition is not less than about 2%. Likewise, the concentration of the saponins in the herbal composition is not less than about 15%. Further, the concentration of polyphenols in the herbal composition is not less than about 2%. Likewise, the concentration of the tannins in the herbal composition is not less than about 3%.

In accordance with the present disclosure, the proanthocyanidins contained in the herbal composition exhibit antioxidant properties and anti-inflammatory properties, activate adenosine monophosphate-activated protein kinase (AMPK), increase glucose transporter (GLUT 4) translocation, improve glucose uptake and insulin sensitivity, and facilitate a reduction in the hepatic fat (liver fat). Further, the saponins contained in the herbal composition bring about a reduction in the absorption of cholesterol in the intestine and modulate hepatic lipid metabolism. Further, the saponins influence the gut-liver axis (in the human body) and reduce fat absorption and modulate blood cholesterol levels, thereby alleviating the symptoms of abnormal weight gain and central obesity. Further, the polyphenols contained in the herbal composition exhibit antioxidant and anti-inflammatory properties. The polyphenols also activate adenosine monophosphate-activated protein kinase (AMPK), thereby increasing glucose uptake and insulin sensitivity, enhancing fat metabolism, promoting fatty acid oxidation, and inhibiting fat synthesis. Furthermore, the polyphenols inhibit carbohydrate-digesting enzymes and thereby lower postprandial glycemia. Furthermore, the polyphenols modulate gut microbiota and positively influence energy balance.

Furthermore, the polyphenols also bring about improvements in certain biomarkers and thereby prevent cell damage and alleviate the symptoms associated with oxidative stress and insulin sensitivity. Further, the tannins contained in the herbal composition also exhibit antioxidant properties and reduce carbohydrate and lipid absorption/digestion. In combination, the proanthocyanidins, polyphenols, saponins, and tannins bring treat and alleviate the symptoms associated with a plurality of metabolic disorders plaguing human beings, by causing a reduction in fasting glucose/HbA1c, improving insulin sensitivity, lowering LDL cholesterol levels and triglyceride levels, enhancing HDL cholesterol levels, reducing fatty liver markers such as Alanine Aminotransferase (ALT) and Aspartate Aminotransferase (AST), and reducing inflammation.

In accordance with the present disclosure, an in vivo efficacy test was performed on five equally distributed groups of black 6 mice (C57BL/6 mice) to determine the anti-obesity effect and weight loss potential of the herbal composition envisaged by the present disclosure. The in-vivo efficacy test was also aimed at determining whether the herbal composition was effective against central obesity. The five equally distributed groups of black 6 mice included only male mice. Each of the five groups contained eight male mice. The mice were divided into five groups based on their body weight. The age of every mouse was 7-8 weeks.

The duration of the in-vivo efficacy test was forty-nine consecutive days. The first group of mice was classified as a normal control group and was fed the chow diet, i.e., a standardized laboratory rodent diet. The second group of mice was classified as a high-fat control group and was fed a high-fat diet (HFD) in a controlled manner. The third, fourth, and fifth groups of mice were fed a high-fat diet (HFD) in a controlled manner along with a dose of the herbal composition.

During the in-vivo efficacy test, the first group of mice, categorized as the normal control group, did not receive the herbal composition envisaged by the present disclosure. Further, the second group of mice also did not receive the herbal composition, but received the high-fat diet (HFD). The third group of mice also received the high-fat diet and was administered a low dose (i.e., 82.20 milligrams (mg) per kilogram (kg) of body weight) of the herbal composition for the period of forty-nine consecutive days, at the same time every day. The fourth group of mice and the fifth group of mice were administered a high dose (i.e., 164.40 milligrams (mg) per kilogram (kg) of body weight) of the herbal composition at the same time every day for the period of forty-nine consecutive days. The volume of the dosage of herbal composition administered to the third, fourth, and fifth groups of mice was 10 mL/kg/body weight/day.

The results achieved by the in-vivo efficacy test demonstrated a marked decrease in the body weight of the mice that were administered the herbal composition in dosages of 82.20 mg/kg and 164.40 mg/kg, respectively (in comparison to the mice that were not administered the herbal composition). Likewise, there was a marked decrease in the total cholesterol level, triglyceride level, and LDL cholesterol levels for the mice that were administered the herbal composition in dosages of 82.20 mg/kg and 164.40 mg/kg, respectively (in comparison to the mice that were not administered the herbal composition). Likewise, there was a marked decrease in the liver weight, subcutaneous fat, and epididymis fat for the mice that were administered the herbal composition in dosages of 82.20 mg/kg and 164.40 mg/kg, respectively (in comparison to the mice that were not administered the herbal composition). Likewise, the mice administered with the herbal composition in dosages of 82.20 mg/kg and 164.40 mg/kg, respectively, showed a marked decrease in the liver function test (LFT)-related parameters, viz., alkaline phosphatase levels, Serum Glutamic-Oxaloacetic Transaminase (SGOT) levels, and Serum Glutamic-Pyruvic Transaminase (SGPT) levels, thereby indicating improved liver health. Likewise, the mice administered with the herbal composition in dosages of 82.20 mg/kg and 164.40 mg/kg, respectively, showed a marked decrease in the levels of tumor necrosis-alpha (TNF-α), a signaling protein that, when synthesized in a higher than desired quantity, promotes inflammation, thereby indicating reduced obesity and inflammation.

In accordance with the present disclosure, a placebo-controlled clinical trial (clinical study) involving human beings as test subjects was also conducted to further establish the efficacy and safety of the herbal composition in treating and alleviating the symptoms associated with metabolic disorders suffered by human beings, including dyslipidemia, stress-induced fatigue, pre-diabetes, type 2 diabetes, high blood sugar levels, elevated blood pressure levels, and abdominal obesity (central obesity).

The clinical trial entailed the participation of forty-five overweight subjects, male and female, in the age group of 20-50. The clinical trial lasted for a period of eighty-four days. The subjects were divided into a treatment group and a placebo group. The treatment group contained twenty-three participants, and the placebo group contained twenty-four participants. The subjects in the treatment group were administered a capsule containing 500 milligrams of the herbal composition every day for sixty consecutive days, half an hour before meals, while the subjects in the placebo group did not receive the herbal composition.

In accordance with the present disclosure, Table 1, provided below, illustrates the changes in the total cholesterol levels observed for each of the subjects in the treatment group and placebo group during the time period (eighty-four days) of the clinical trial. In accordance with the present disclosure, Table 1 illustrates the total cholesterol levels across eighty-four days in each of the subjects who participated in the clinical trial (in the treatment group and the placebo group). All values in Table 1 are represented using the expression ‘mean±SD’, with the sample size (n) of the placebo group being twenty-two and the sample size (n) of the treatment group being twenty-three. Typically, the expression ‘mean±standard deviation (mean±SD)’ depicts the central value (the mean) along with a measure of the spread or variability (the standard deviation).

In accordance with the present disclosure, the values of ‘mean±SD’ for the treatment group were statistically compared with the ‘mean±SD’ values for the placebo group for day 0 (baseline; beginning of the clinical study), day 42, and day 84 (end of the clinical study). As illustrated in Table 1, the ‘mean±SD’ values for the treatment group illustrated a reasonable decrease in the total cholesterol levels vis-à-vis the placebo group that was not administered the herbal composition.

TABLE 1
Total Cholesterol Levels

| | |
|---|---|
| All the values are | Total Cholesterol (mg/dL) |

| represented as | Herbal Composition | |
|---|---|---|
| ‘mean ± SD’ | (Treatment Group) | Placebo Group |
| Baseline | 180.78 ± 29.90 | 180.50 ± 25.05 |
| Day 42 | 174.29 ± 24.81 | 185.89 ± 36.52 |
| Day 84 | 176.33 ± 27.77 | 189.68 ± 31.93 |
| Change | −6.50 ± 38.07 | 5.09 ± 46.19 |
| Baseline vs. Day 42 | | |
| Change | −4.45 ± 45.32 | 9.18 ± 43.33 |
| Baseline vs. Day 84 | | |

From the total cholesterol level-related changes tabulated in Table 1, it is apparent that the herbal composition causes a reasonable reduction (decrease) in the total cholesterol levels in the subjects of the treatment group (baseline versus day 42: −6.50±38.07 and baseline versus day 84: −4.45±45.32) who were administered the herbal composition daily for sixty days, thereby implying a positive impact as far as the reduction in the total cholesterol levels is concerned.

On the contrary, the subjects in the placebo group had an increase in the total cholesterol levels (baseline versus day 42: 5.09±46.19 and baseline versus day 84: 9.18±43.33). Therefore, from Table 1, it is apparent that the herbal composition envisaged by the present disclosure brings about a reasonable reduction (decrease) in the total cholesterol levels for the subjects who were administered the prescribed dosage of the herbal composition for sixty days.

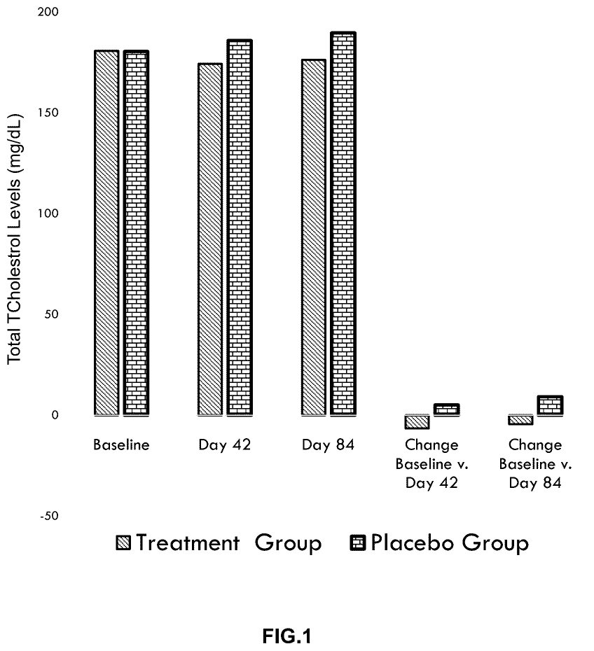

In accordance with the present disclosure, FIG. 1 illustrates a clustered column graph that describes the changes to the total cholesterol levels of the human subjects on day 0 (baseline), day 42, and day 84 of the clinical study. In FIG. 1, the changes to the total cholesterol levels are plotted on the vertical axis (y-axis), and the days on which the total cholesterol level-related readings were taken are indicated on the horizontal axis (x-axis). In accordance with the present disclosure, FIG. 1 is a graphical representation of the total cholesterol level-related readings illustrated in Table 1.

In accordance with the present disclosure, Table 2, provided below, illustrates the changes in the total triglyceride levels observed for each of the subjects in the treatment group and placebo group during the time period (eighty-four days) when the clinical trial was conducted. The triglyceride levels were measured on day 0 (baseline; beginning of the clinical study), day 42, and day 84 (end of the clinical study).

TABLE 2
Triglyceride Levels

| | |
|---|---|
| All the values are | Triglyceride Levels (mg/dL) |

| represented as | Herbal Composition | |
|---|---|---|
| ‘mean ± SD’ | (Treatment Group) | Placebo Group |
| Baseline | 140.39 ± 34.85 | 144.18 ± 38.51 |
| Day 42 | 138.39 ± 37.12 | 147.50 ± 44.51 |
| Day 84 | 134.91 ± 45.48 | 147.96 ± 37.48 |
| Change | −2.00 ± 46.33 | 3.32 ± 56.21 |
| Baseline vs. Day 42 | | |
| Change | −5.48 ± 48.25 | 3.77 ± 52.04 |
| Baseline vs. Day 84 | | |

In accordance with the present disclosure, the values of ‘mean±SD’ for the treatment group were statistically compared with the ‘mean±SD’ values for the placebo group for days 0, 42, and 84. As illustrated in Table 2, the ‘mean±SD’ values of the treatment group showed a reasonable decrease in terms of triglyceride levels vis-à-vis the placebo group that was not administered the herbal composition.

From the triglyceride level-related changes tabulated in Table 2, it is apparent that the herbal composition did cause a reasonable reduction (decrease) in the triglyceride levels in the subjects of the treatment group (baseline versus day 42: −2.00±46.33 and baseline versus day 84: −5.48±48.25) who were administered the herbal composition daily for sixty days, thereby implying a positive impact as far as the reduction in the triglyceride levels is concerned.

On the contrary, the subjects in the placebo group had an increase in the triglyceride levels (baseline versus day 42: 3.32±56.21 and baseline versus day 84: 3.77±52.04). Therefore, from Table 2, it is apparent that the herbal composition envisaged by the present disclosure brings about a reasonable reduction (decrease) in the triglyceride levels for the subjects who were administered the prescribed dosage of the herbal composition for sixty days.

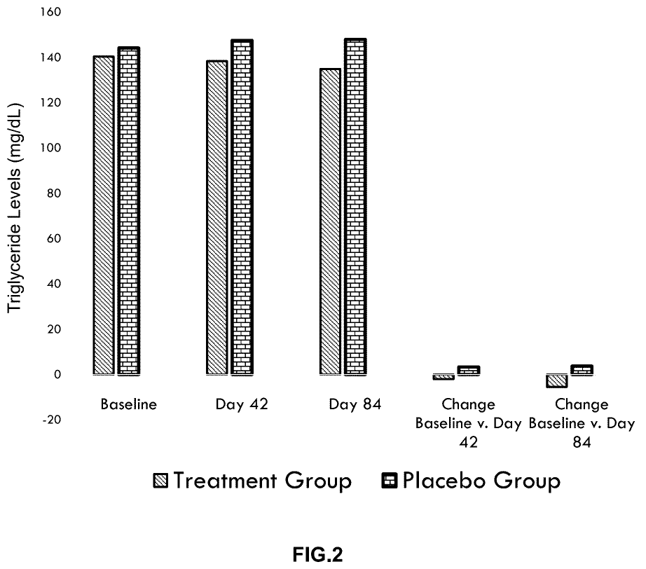

In accordance with the present disclosure, FIG. 2 illustrates a clustered column graph that describes the changes to the triglyceride levels of the human subjects on day 0 (baseline), day 42, and day 84 of the clinical study. In FIG. 2, the changes to the triglyceride levels are plotted on the vertical axis (y-axis), and the days on which the triglyceride level-related readings were taken are indicated on the horizontal axis (x-axis). In accordance with the present disclosure, FIG. 2 is a graphical representation of the triglyceride level-related readings illustrated in Table 2.

In accordance with the present disclosure, Table 3, provided below, illustrates the changes in the LDL cholesterol levels observed for each of the subjects in the treatment group and placebo group during the time period (eighty-four days) when the clinical trial was conducted.

In accordance with the present disclosure, the values of ‘mean±SD’ for the treatment group were statistically compared with the ‘mean±SD’ values for the placebo group for days 0, 42, and 84. As illustrated in Table 3, the ‘mean±SD’ values of the treatment group showed a reasonable decrease in terms of the LDL cholesterol levels vis-à-vis the placebo group that was not administered the herbal composition.

TABLE 3
Low-Density Lipoprotein (LDL) Levels

| | |
|---|---|
| All the values are | LDL Cholesterol Levels (mg/dL) |

| represented as | Herbal Composition | |
|---|---|---|
| ‘mean ± SD’ | (Treatment Group) | Placebo Group |
| Baseline | 115.61 ± 27.71 | 114.73 ± 22.75 |
| Day 42 | 108.91 ± 23.12 | 108.55 ± 43.42 |
| Day 84 | 104.61 ± 32.71 | 121.59 ± 27.80 |
| Change | −6.70 ± 37.50 | −6.18 ± 48.97 |
| Baseline vs. Day 42 | | |
| Change | −11.00 ± 51.62 | 6.86 ± 38.81 |
| Baseline vs. Day 84 | | |

From the LDL cholesterol level-related changes tabulated in Table 3, it is apparent that the herbal composition causes a reasonable reduction (decrease) in the LDL cholesterol levels in the subjects of the treatment group (baseline versus day 42: −6.70±37.50 and baseline versus day 84: −11.00±51.62) who were administered the herbal composition daily for sixty days, thereby implying a positive impact as far as the reduction in the LDL cholesterol levels is concerned.

On the contrary, the subjects in the placebo group had an increase in the LDL cholesterol levels on day 84 (6.86±38.81), despite showing a decrease (−6.18±48.97) in the LDL cholesterol levels on day 42. Therefore, from Table 3, it is apparent that the herbal composition envisaged by the present disclosure brings about a reasonable reduction (decrease) in the LDL cholesterol levels for the subjects who were administered the prescribed dosage of the herbal composition for sixty days.

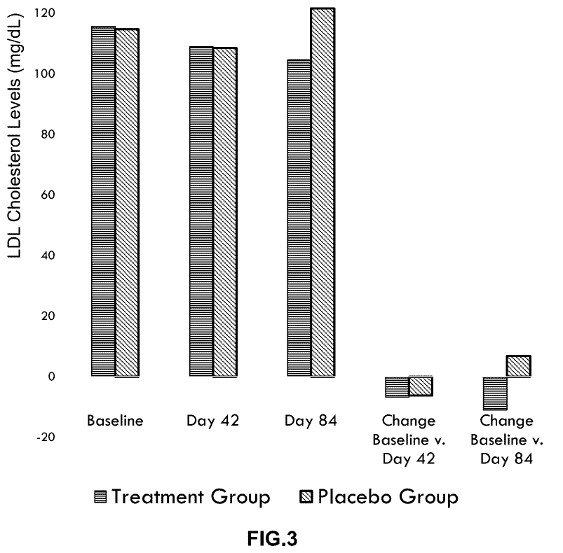

In accordance with the present disclosure, FIG. 3 illustrates a clustered column graph that describes the changes in the LDL cholesterol levels for the human subjects on day 0 (baseline), day 42, and day 84 of the clinical study. In FIG. 3, the changes to the LDL cholesterol levels are plotted on the vertical axis (y-axis), and the days on which the LDL cholesterol level-related readings were taken are indicated on the horizontal axis (x-axis). In accordance with the present disclosure, FIG. 3 is a graphical representation of the LDL cholesterol levels-related readings illustrated in Table 3.

In accordance with the present disclosure, Table 4, provided below, illustrates the changes in the HDL cholesterol levels observed for each of the subjects in the treatment group and placebo group during the time period (eighty-four days) when the clinical trial was conducted.

TABLE 4
High-Density Lipoprotein (HDL) Levels

| | |
|---|---|
| All the values are | HDL Cholesterol Levels (mg/dL) |

| represented as | Herbal Composition | |
|---|---|---|
| ‘mean ± SD’ | (Treatment Group) | Placebo Group |
| Baseline | 37.09 ± 6.67 | 37.05 ± 6.31 |
| Day 42 | 37.70 ± 6.56 | 47.55 ± 22.83 |
| Day 84 | 44.74 ± 21.69 | 38.50 ± 8.32 |
| Change | 0.61 ± 9.46 | 10.50 ± 22.91 |
| Baseline vs. Day 42 | | |
| Change | 7.65 ± 23.53 | 1.46 ± 10.55 |
| Baseline vs. Day 84 | | |

In accordance with the present disclosure, the values of ‘mean±SD’ for the treatment group were statistically compared with the ‘mean±SD’ values for the placebo group for days 0, 42, and 84. As illustrated in Table 4, the ‘mean±SD’ values of the treatment group showed a reasonable increase in terms of the HDL cholesterol levels vis-à-vis the placebo group that was not administered the herbal composition. The ‘mean±SD’ values for the placebo group did show an increase in terms of the HDL cholesterol levels, but the increase in the HDL cholesterol levels for the treatment group was consistently and significantly higher than the increase in the HDL cholesterol levels for the placebo group.

From the HDL cholesterol levels-related changes tabulated in Table 4, it is apparent that the herbal composition did cause a reasonable increase in the HDL cholesterol levels in the subjects of the treatment group (baseline versus day 84: 7.65±23.53) who were administered the herbal composition daily for sixty days, thereby implying a positive impact as far as the enhancement of the HDL cholesterol levels is concerned. Further, the subjects in the placebo group also witnessed an increase in the HDL cholesterol levels (baseline versus day 42: 10.50±22.91 and baseline versus day 84: 1.46±10.55). However, the positive change (the increase in the HDL cholesterol levels) witnessed for the treatment group is consistently and significantly higher than the positive change witnessed for the placebo group. Therefore, from Table 4, it is apparent that the herbal composition envisaged by the present disclosure brings about a reasonable increase in the HDL cholesterol levels for the subjects who were administered the prescribed dosage of the herbal composition for sixty days.

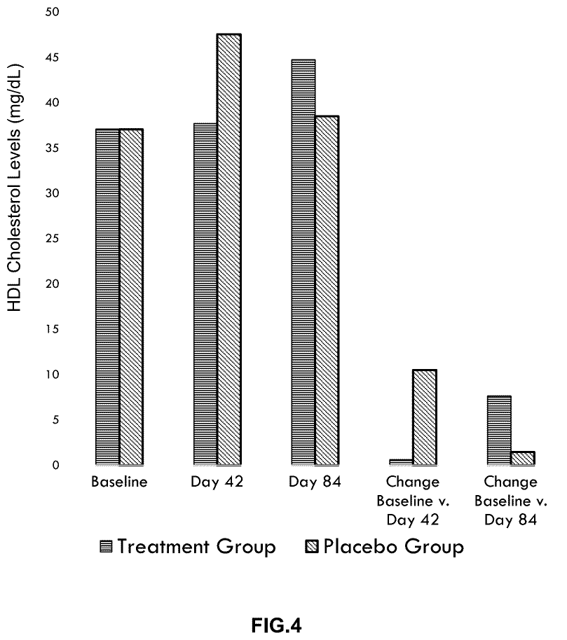

In accordance with the present disclosure, FIG. 4 illustrates a clustered column graph that describes the changes in the HDL cholesterol levels for the human subjects on day 0 (baseline), day 42, and day 84 of the clinical study. In FIG. 4, the changes to the HDL cholesterol levels are plotted on the vertical axis (y-axis), and the days on which the HDL cholesterol level-related readings were taken are indicated on the horizontal axis (x-axis). In accordance with the present disclosure, FIG. 4 is a graphical representation of the HDL cholesterol levels-related readings illustrated in Table 4.

In accordance with the present disclosure, Table 5, provided below, illustrates the changes in the adiponectin levels observed for each of the subjects in the treatment group and placebo group during the time period (eighty-four days) when the clinical trial was conducted.

TABLE 5
Adiponectin Levels

| | |
|---|---|
| All the values are | Adiponectin Levels (mg/dL) |

| represented as | Herbal Composition | |
|---|---|---|
| ‘mean ± SD’ | (Treatment Group) | Placebo Group |
| Baseline | 5.38 ± 0.68 | 5.42 ± 0.54 |
| Day 42 | 6.01 ± 0.76 | 5.52 ± 0.94 |
| Day 84 | 6.29 ± 1.02 | 5.84 ± 1.20 |
| Change | 0.63 ± 0.74 | 0.10 ± 0.71 |
| Baseline vs. Day 42 | | |
| Change | 0.91 ± 0.91 | 0.42 ± 1.02 |
| Baseline vs. Day 84 | | |

In accordance with the present disclosure, the values of ‘mean±SD’ for the treatment group were statistically compared with the ‘mean±SD’ values for the placebo group for days 0, 42, and 84. As illustrated in Table 5, the ‘mean±SD’ values of the treatment group showed a reasonable increase in terms of the adiponectin levels vis-à-vis the placebo group that was not administered the herbal composition. The ‘mean±SD’ values for the placebo group did show an increase in terms of the adiponectin levels, but the increase in the adiponectin levels for the treatment group was consistently and significantly higher than the increase in the adiponectin levels for the placebo group.

From the adiponectin level-related changes tabulated in Table 5, it is apparent that the herbal composition did cause a reasonable increase in the adiponectin levels in the subjects of the treatment group (baseline versus day 42: 0.63±0.74 and baseline versus day 84: 0.91±0.91) who were administered the herbal composition daily for sixty days, thereby implying a positive impact as far as the enhancement of the adiponectin levels is concerned. Further, the subjects in the placebo group also witnessed an increase in the adiponectin levels (baseline versus day 42: 0.10±0.71 and baseline versus day 84: 0.42±1.02).

However, the positive change (the increase in the adiponectin levels) witnessed for the treatment group is consistently and significantly higher than the positive change witnessed for the placebo group. Therefore, from Table 5, it is apparent that the herbal composition envisaged by the present disclosure brings about a reasonable increase in the adiponectin levels for the subjects who were administered the prescribed dosage of the herbal composition for sixty days.

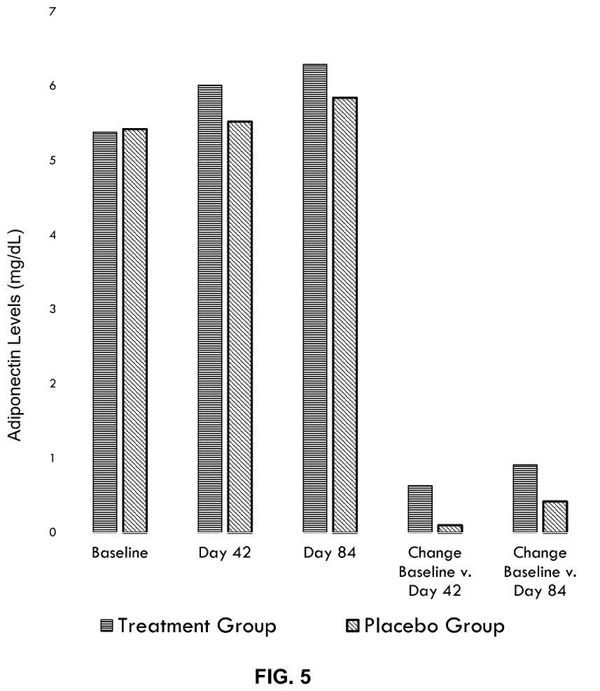

In accordance with the present disclosure, FIG. 5 illustrates a clustered column graph that describes the changes in the adiponectin levels for the human subjects on day 0 (baseline), day 42, and day 84 of the clinical study. In FIG. 5, the changes to the adiponectin levels are plotted on the vertical axis (y-axis), and the days on which the adiponectin level-related readings were taken are indicated on the horizontal axis (x-axis). In accordance with the present disclosure, FIG. 5 is a graphical representation of the adiponectin levels-related readings illustrated in Table 5.

In accordance with the present disclosure, Table 6, provided below, illustrates the changes to the body fat percentage observed for each of the subjects in the treatment group and placebo group during the time period (eighty-four days) when the clinical trial was conducted. In accordance with the present disclosure, the values of ‘mean±SD’ for the treatment group were statistically compared with the ‘mean±SD’ values for the placebo group for days 0, 42, and 84.

TABLE 6
Body Fat Percentage

| | |
|---|---|
| All the values are | Body Fat (Percentage) |

| represented as | Herbal Composition | |
|---|---|---|
| ‘mean ± SD’ | (Treatment Group) | Placebo Group |
| Baseline | 32.87 ± 5.49 | 35.12 ± 5.59 |
| Day 42 | 32.66 ± 5.48 | 35.28 ± 5.46 |
| Day 84 | 32.59 ± 5.73 | 35.46 ± 5.51 |
| Change | −0.21 ± 0.63 | 0.16 ± 0.81 |
| Baseline vs. Day 42 | | |
| Change | −0.28 ± 0.98 | 0.34 ± 0.90 |
| Baseline vs. Day 84 | | |

As illustrated in Table 6, the ‘mean±SD’ values of the treatment group showed a reasonable decrease in terms of the body fat percentage vis-à-vis the placebo group that was not administered the herbal composition. Further, the ‘mean±SD’ values for the placebo group showed an increase in terms of the body fat percentage.

From the body fat percentage-related changes tabulated in Table 6, it is apparent that the herbal composition causes a reasonable reduction (decrease) in the body fat percentage of the subjects of the treatment group (baseline versus day 42: −0.21±0.63 and baseline versus day 84: −0.28±0.98) who were administered the herbal composition daily for sixty days, thereby implying a positive impact as far as the reduction of the body fat is concerned. On the contrary, as illustrated in Table 6, the subjects in the placebo group witnessed an increase in the body fat percentage (baseline versus day 42: 0.16±0.81 and baseline versus day 84: 0.34±0.90). Therefore, from Table 6, it is apparent that the herbal composition envisaged by the present disclosure brings about a reasonable decrease in the body fat percentage for the subjects who were administered the prescribed dosage of the herbal composition for sixty days.

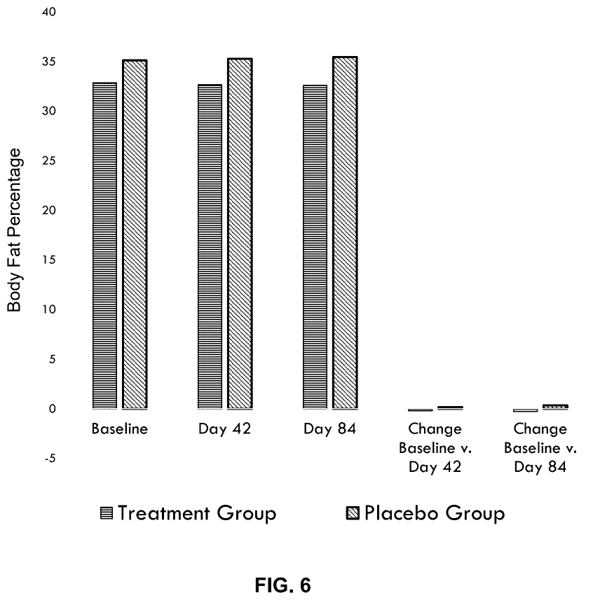

In accordance with the present disclosure, FIG. 6 illustrates a clustered column graph that describes the changes in the body fat percentage for the human subjects on day 0 (baseline), day 42, and day 84 of the clinical study. In FIG. 6, the changes to the body fat percentage are plotted on the vertical axis (y-axis), and the days on which the body fat percentage-related readings were taken are indicated on the horizontal axis (x-axis). In accordance with the present disclosure, FIG. 6 is a graphical representation of the body fat percentage-related readings illustrated in Table 6.

In accordance with the present disclosure, Table 7, provided below, illustrates the lean mass percentage observed for each of the subjects in the treatment group and placebo group during the time period (eighty-four days) when the clinical trial was conducted.

In accordance with the present disclosure, the values of ‘mean±SD’ for the treatment group were statistically compared with the ‘mean±SD’ values for the placebo group for days 0, 42, and 84. As illustrated in Table 7, the ‘mean±SD’ values of the treatment group showed a reasonable increase in terms of the lean mass percentage vis-à-vis the placebo group that was not administered the herbal composition. Further, the ‘mean±SD’ values for the placebo group showed a decrease in terms of the lean mass percentage, complementing the increase in the body fat percentage previously illustrated in Table 6.

TABLE 7
Lean Mass Percentage

| | |
|---|---|
| All the values are | Lean Mass (Percentage) |

| represented as | Herbal Composition | |
|---|---|---|
| ‘mean ± SD’ | (Treatment Group) | Placebo Group |
| Baseline | 64.33 ± 5.49 | 62.05 ± 5.59 |
| Day 42 | 64.53 ± 5.47 | 61.89 ± 5.46 |
| Day 84 | 64.60 ± 5.70 | 61.71 ± 5.51 |
| Change | 0.21 ± 0.63 | −0.16 ± 0.81 |
| Baseline vs. Day 42 | | |
| Change | 0.28 ± 0.98 | −0.34 ± 0.90 |
| Baseline vs. Day 84 | | |

As illustrated in Table 7, the ‘mean±SD’ values of the treatment group showed a reasonable increase in the lean body mass percentage vis-à-vis the placebo group that was not administered the herbal composition. Further, the ‘mean±SD’ values for the placebo group showed a decrease in the lean body mass percentage.

From the lean body mass percentage-related changes tabulated in Table 7, it is apparent that the herbal composition causes a reasonable increase (enhancement) in the lean body mass percentage of the subjects of the treatment group (baseline versus day 42: 0.21±0.63 and baseline versus day 84: 0.28±0.98) who were administered the herbal composition daily for sixty days, thereby implying a positive impact as far as the enhancement of lean body mass is concerned. On the contrary, as illustrated in Table 7, the subjects in the placebo group witnessed a decrease in the lean body mass (baseline versus day 42: −0.16±0.81 and baseline versus day 84: −0.34±0.90). Therefore, from Table 7, it is apparent that the herbal composition envisaged by the present disclosure brings about a reasonable increase in the lean body mass for the subjects who were administered the prescribed dosage of the herbal composition for sixty days.

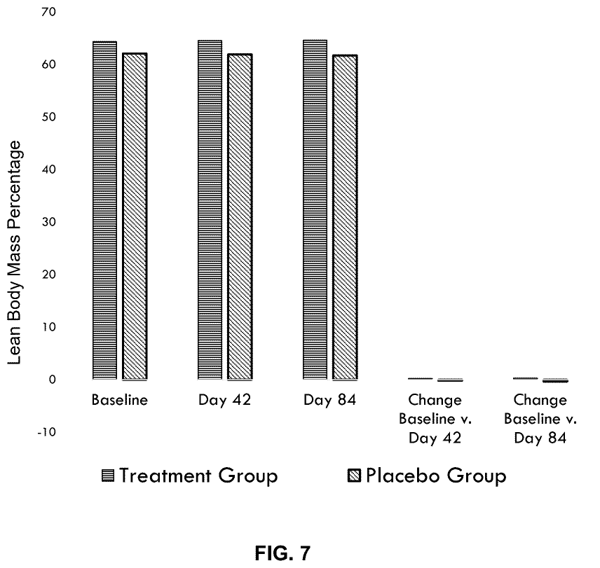

In accordance with the present disclosure, FIG. 7 illustrates a clustered column graph that describes the changes in the lean body mass percentage of the human subjects on day 0 (baseline), day 42, and day 84 of the clinical study. In FIG. 7, the changes to the lean body mass percentage are plotted on the vertical axis (y-axis), and the days on which the lean body mass percentage-related readings were taken are indicated on the horizontal axis (x-axis). In accordance with the present disclosure, FIG. 7 is a graphical representation of the lean body mass percentage-related readings illustrated in Table 7.

In accordance with the present disclosure, Table 8, provided below, illustrates the changes in the waist circumference observed for each of the subjects in the treatment group and placebo group during the time period (eighty-four days) when the clinical trial was conducted. In accordance with the present disclosure, the values of ‘mean±SD’ for the treatment group were statistically compared with the ‘mean±SD’ values for the placebo group for days 0, 42, and 84. As illustrated in Table 8, the ‘mean±SD’ values of the treatment group showed a reasonable decrease in terms of the waist circumference vis-à-vis the placebo group that was not administered the herbal composition. Further, the ‘mean±SD’ values for the placebo group showed an increase in terms of the waist circumference, complementing the increase in the body fat percentage (Table 6) and the reduction in the lean body mass (Table 7).

TABLE 8
Waist Circumference

| | |
|---|---|
| All the values are | Waist Circumference (Centimetres) |

| represented as | Herbal Composition | |
|---|---|---|
| ‘mean ± SD’ | (Treatment Group) | Placebo Group |
| Baseline | 94.54 ± 5.82 | 92.98 ± 6.64 |
| Day 42 | 93.88 ± 5.25 | 93.81 ± 6.48 |
| Day 84 | 93.71 ± 5.53 | 95.24 ± 6.74 |
| Change | −0.67 ± 3.90 | 0.83 ± 3.04 |
| Baseline vs. Day 42 | | |
| Change | −0.83 ± 3.60 | 2.26 ± 3.33 |
| Baseline vs. Day 84 | | |

As illustrated in Table 8, the ‘mean±SD’ values of the treatment group showed a reasonable decrease in terms of the waist circumference vis-à-vis the placebo group that was not administered the herbal composition. Further, the ‘mean±SD’ values for the placebo group showed an increase in the waist circumference.

From the waist circumference-related changes tabulated in Table 8, it is apparent that the herbal composition did cause a reasonable reduction in the waist circumference of the subjects of the treatment group (baseline versus day 42: −0.67±3.90 and baseline versus day 84: −0.83±3.60) who were administered the herbal composition daily for sixty days, thereby implying a positive impact as far as the reduction of the waist circumference is concerned. On the contrary, as illustrated in Table 8, the subjects in the placebo group witnessed a reasonable increase in the circumference of the waistline (baseline versus day 42: 0.83±3.04 and baseline versus day 84: 2.26±3.33). Therefore, from Table 8, it is apparent that the herbal composition envisaged by the present disclosure brings about a reasonable reduction in the circumference of the waistline for the subjects who were administered the prescribed dosage of the herbal composition for sixty days.

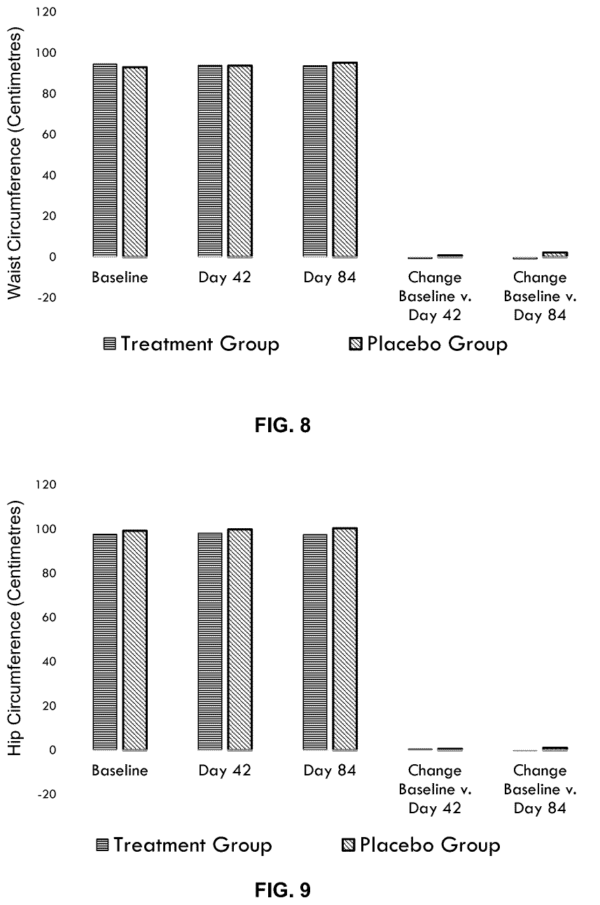

In accordance with the present disclosure, FIG. 8 illustrates a clustered column graph that describes the changes to the waist circumference of the human subjects on day 0 (baseline), day 42, and day 84 of the clinical study. In FIG. 8, the changes to the waist circumference are plotted on the vertical axis (y-axis), and the days on which the waist circumference-related readings were taken are indicated on the horizontal axis (x-axis). In accordance with the present disclosure, FIG. 8 is a graphical representation of the waist circumference-related readings illustrated in Table 8.

In accordance with the present disclosure, Table 9, provided below, illustrates the changes in the hip circumference observed for each of the subjects in the treatment group and placebo group during the time period (eighty-four days) when the clinical trial was conducted. In accordance with the present disclosure, the values of ‘mean±SD’ for the treatment group were statistically compared with the ‘mean±SD’ values for the placebo group for days 0, 42, and 84. As illustrated in Table 9, the ‘mean±SD’ values of the treatment group showed a reasonable decrease in terms of the hip circumference vis-à-vis the placebo group that was not administered the herbal composition. Further, the ‘mean±SD’ values for the placebo group showed an increase in terms of the hip circumference, complementing the increase in the body fat percentage (Table 6), the reduction in the lean body mass (Table 7), and the increase in the waist circumference (Table 8).

TABLE 9
Hip Circumference

| | |
|---|---|
| All the values are | Hip Circumference (Centimetres) |

| represented as | Herbal Composition | |
|---|---|---|
| ‘mean ± SD’ | (Treatment Group) | Placebo Group |
| Baseline | 97.67 ± 5.87 | 99.24 ± 5.48 |
| Day 42 | 98.13 ± 5.14 | 99.91 ± 5.38 |
| Day 84 | 97.50 ± 5.24 | 100.36 ± 5.64 |
| Change | 0.46 ± 4.14 | 0.67 ± 3.15 |
| Baseline vs. Day 42 | | |
| Change | −0.17 ± 3.62 | 1.12 ± 3.94 |
| Baseline vs. Day 84 | | |

As illustrated in Table 9, the ‘mean±SD’ values of the treatment group showed a reasonable decrease in terms of the hip circumference vis-à-vis the placebo group that was not administered the herbal composition. Further, the ‘mean SSD’ values for the placebo group showed an increase in the hip circumference.

From the hip circumference-related changes tabulated in Table 9, it is apparent that the herbal composition did cause a reasonable reduction in the hip circumference of the subjects of the treatment group (baseline versus day 84: −0.17±3.62) who were administered the herbal composition daily for sixty days, thereby implying a positive impact as far as the reduction of the hip circumference is concerned. On the contrary, as illustrated in Table 9, the subjects in the placebo group witnessed a reasonable increase in the hip circumference (baseline versus day 42: 0.67±3.15 and baseline versus day 84: 1.12±3.94). Therefore, from Table 9, it is apparent that the herbal composition envisaged by the present disclosure brings about a reasonable reduction in the hip circumference for the subjects who were administered the prescribed dosage of the herbal composition for sixty days.

In accordance with the present disclosure, FIG. 9 illustrates a clustered column graph that describes the changes to the hip circumference of the human subjects on day 0 (baseline), day 42, and day 84 of the clinical study. In FIG. 9, the changes to the hip circumference are plotted on the vertical axis (y-axis), and the days on which the hip circumference-related readings were taken are indicated on the horizontal axis (x-axis). In accordance with the present disclosure, FIG. 9 is a graphical representation of the hip circumference-related readings illustrated in Table 9.

In accordance with the present disclosure, Table 10, provided below, illustrates the changes in the waist-to-hip ratio (WHR) observed for each of the subjects in the treatment group and placebo group during the time period (eighty-four days) when the clinical trial was conducted. In accordance with the present disclosure, the values of ‘mean±SD’ for the treatment group were statistically compared with the ‘mean±SD’ values for the placebo group for days 0, 42, and 84.

TABLE 10
Waist-to-Hip Ratio

| | |
|---|---|
| All the values are | Waist-to-Hip Ratio |

| represented as | Herbal Composition | |
|---|---|---|
| ‘mean ± SD’ | (Treatment Group) | Placebo Group |
| Baseline | 0.97 ± 0.05 | 0.94 ± 0.06 |
| Day 42 | 0.96 ± 0.05 | 0.94 ± 0.05 |
| Day 84 | 0.96 ± 0.05 | 0.95 ± 0.07 |
| Change | −0.01 ± 0.05 | 0.00 ± 0.05 |
| Baseline vs. Day 42 | | |
| Change | −0.01 ± 0.04 | 0.01 ± 0.03 |
| Baseline vs. Day 84 | | |

As illustrated in Table 10, the ‘mean±SD’ values of the treatment group showed a decrease (reduction) in the waist-to-hip ratio vis-à-vis the placebo group that was not administered the herbal composition. Further, the ‘mean±SD’ values for the placebo group showed an increase in terms of the waist-to-hip ratio, complementing the increase in the body fat percentage (Table 6), the reduction in the lean body mass (Table 7), the increase in the waist circumference (Table 8), and the increase in the hip circumference (Table 9).

From the waist-to-hip ratio-related changes tabulated in Table 10, it is apparent that the herbal composition did cause a reduction in the waist-to-hip ratio for the subjects in the treatment group (baseline versus day 42: −0.01±0.05 and baseline versus day 84: −0.01±0.04) who were administered the herbal composition daily for sixty days, thereby implying a positive impact as far as the reduction of the waist-to-hip ratio is concerned. Further, as illustrated in Table 10, the subjects in the placebo group witnessed no changes in the waist-to-hip ratio on day 42 and a marginal increase in the waist-to-hip ratio on day 84 (baseline versus day 84: 0.01±0.03). Therefore, from Table 10, it is apparent that the herbal composition envisaged by the present disclosure brings about a marginal reduction in the waist-to-hip ratio for the subjects who were administered the prescribed dosage of the herbal composition for sixty days.

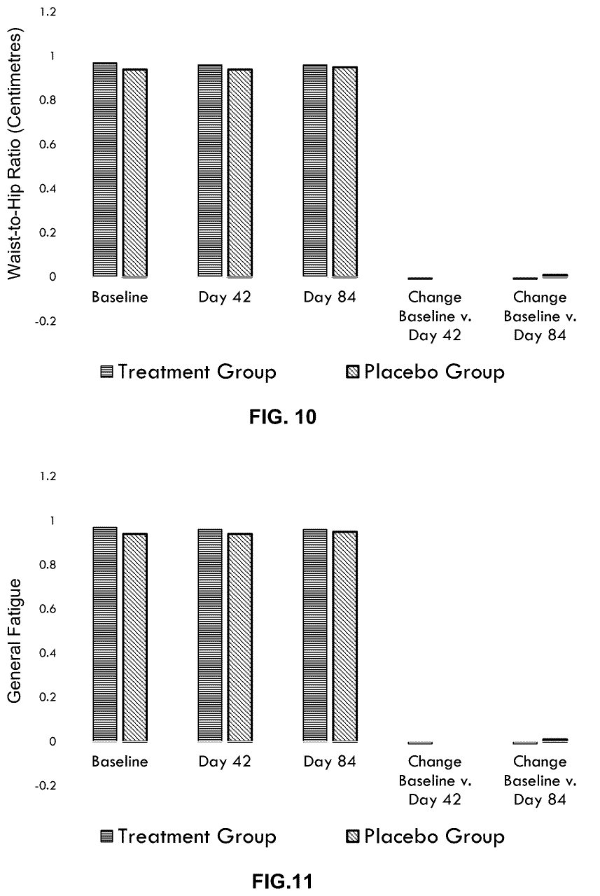

In accordance with the present disclosure, FIG. 10 illustrates a clustered column graph that describes the changes to the waist-to-hip ratio of the human subjects on day 0 (baseline), day 42, and day 84 of the clinical study. In FIG. 10, the changes to the waist-to-hip ratio are plotted on the vertical axis (y-axis), and the days on which the waist-to-hip ratio-related readings were taken are indicated on the horizontal axis (x-axis). In accordance with the present disclosure, FIG. 10 is a graphical representation of the waist-to-hip ratio-related readings illustrated in Table 10.

In accordance with the present disclosure, Table 11, provided below, illustrates the changes in the general fatigue score observed for each of the subjects in the treatment group and placebo group during the time period (eighty-four days) when the clinical trial was conducted. In accordance with the present disclosure, the values of ‘mean±SD’ for the treatment group were statistically compared with the ‘mean±SD’ values for the placebo group for days 0, 42, and 84. As illustrated in Table 11, the ‘mean±SD’ values of the treatment group showed a reasonable decrease (reduction) in the general fatigue-related score vis-à-vis the placebo group that was not administered the herbal composition, thereby implying a significant reduction in the general fatigue levels of the subjects who were administered the prescribed dose of the herbal composition, and confirming the therapeutic efficacy of the herbal composition in terms of alleviating and treating general fatigue-related symptoms. Further, the ‘mean±SD’ values for the placebo group showed an increase in terms of the general fatigue score.

TABLE 11
General Fatigue Score

| | |
|---|---|
| All the values are | General Fatigue |

| represented as | Herbal Composition | |
|---|---|---|
| ‘mean ± SD’ | (Treatment Group) | Placebo Group |
| Baseline | 11.88 ± 1.75 | 11.81 ± 0.06 |
| Day 42 | 10.67 ± 1.40 | 11.81 ± 0.05 |
| Day 84 | 10.46 ± 1.32 | 11.86 ± 0.07 |
| Change | −1.21 ± 1.18 | 0.00 ± 0.45 |
| Baseline vs. Day 42 | | |
| Change | −1.42 ± 1.18 | 0.05 ± 0.50 |
| Baseline vs. Day 84 | | |

From the general fatigue score-related changes tabulated in Table 11, it is apparent that the herbal composition did cause a reduction in the general fatigue score (thereby implying a reasonable reduction in fatigue levels) for the subjects in the treatment group (baseline versus day 42: −1.21±1.18 and baseline versus day 84: −1.42±1.18) who were administered the herbal composition daily for sixty days. Further, as illustrated in Table 11, the subjects in the placebo group witnessed no changes in the general fatigue score on day 42 and a marginal increase in the general fatigue score on day 84 (baseline versus day 84: 0.05±0.50). However, it is pertinent to note that while the subjects belonging to the treatment group witnessed a significant decrease in the general fatigue score (treatment group—baseline versus day 42: −1.21±1.18 and baseline versus day 84: −1.42±1.18), the subjects in the placebo group witnessed no such significant reduction but a marginal increase in the general fatigue score. Therefore, from Table 11, it is apparent that the herbal composition envisaged by the present disclosure brings about a reduction in the general fatigue score, thereby implying a reasonable reduction in fatigue levels for the subjects who were administered the prescribed dosage of the herbal composition for sixty days.

In accordance with the present disclosure, FIG. 11 illustrates a clustered column graph that describes the changes to the general fatigue-related scores of the human subjects on day 0 (baseline), day 42, and day 84 of the clinical study. In FIG. 11, the changes to the general fatigue-related scores are plotted on the vertical axis (y-axis), and the days on which the general fatigue-related scores were determined are indicated on the horizontal axis (x-axis). In accordance with the present disclosure, FIG. 11 is a graphical representation of the general fatigue-related scores illustrated in Table 11.

In accordance with the present disclosure, Table 12, provided below, illustrates the changes in the physical fatigue scores observed for each of the subjects in the treatment group and placebo group during the time period (eighty-four days) when the clinical trial was conducted. As illustrated in Table 12, the ‘mean±SD’ values of the treatment group showed a reasonable decrease (reduction) in the physical fatigue-related score vis-à-vis the placebo group that was not administered the herbal composition, thereby implying a significant reduction in the physical fatigue levels of the subjects who were administered the prescribed dose of the herbal composition, and confirming the therapeutic efficacy of the herbal composition in terms of alleviating and treating physical fatigue-related symptoms.

TABLE 12
Physical Fatigue Score

| | |
|---|---|
| All the values are | Physical Fatigue |

| represented as | Herbal Composition | |
|---|---|---|
| ‘mean ± SD’ | (Treatment Group) | Placebo Group |
| Baseline | 10.92 ± 1.18 | 10.81 ± 1.29 |
| Day 42 | 10.13 ± 1.26 | 11.38 ± 1.36 |
| Day 84 | 9.88 ± 1.33 | 11.43 ± 1.43 |
| Change | −0.79 ± 0.78 | 0.57 ± 0.98 |
| Baseline vs. Day 42 | | |
| Change | −1.04 ± 0.69 | 0.62 ± 0.97 |
| Baseline vs. Day 84 | | |

From the physical fatigue score-related changes tabulated in Table 12, it is apparent that the herbal composition causes a reduction in the physical fatigue score for the subjects in the treatment group (baseline versus day 42: −0.79±0.78 and baseline versus day 84: −1.04±0.69) who were administered the herbal composition daily for sixty days. Further, as illustrated in Table 12, the subjects in the placebo group witnessed a reasonable increase in the physical fatigue score on day 42 (0.57±0.98) and yet another marginal increase in the physical fatigue score on day 84 (0.62±0.97).

However, it is pertinent to note that while the subjects belonging to the treatment group witnessed a significant decrease in the physical fatigue score (treatment group—baseline versus day 42: −0.79±0.78 and baseline versus day 84: −1.04±0.69), the subjects in the placebo group witnessed no such significant reduction but an increase in the physical fatigue score. Therefore, from Table 12, it is apparent that the herbal composition envisaged by the present disclosure brings about a reduction in the physical fatigue score, thereby implying a reasonable reduction in fatigue levels for the subjects who were administered the prescribed dosage of the herbal composition for sixty days.

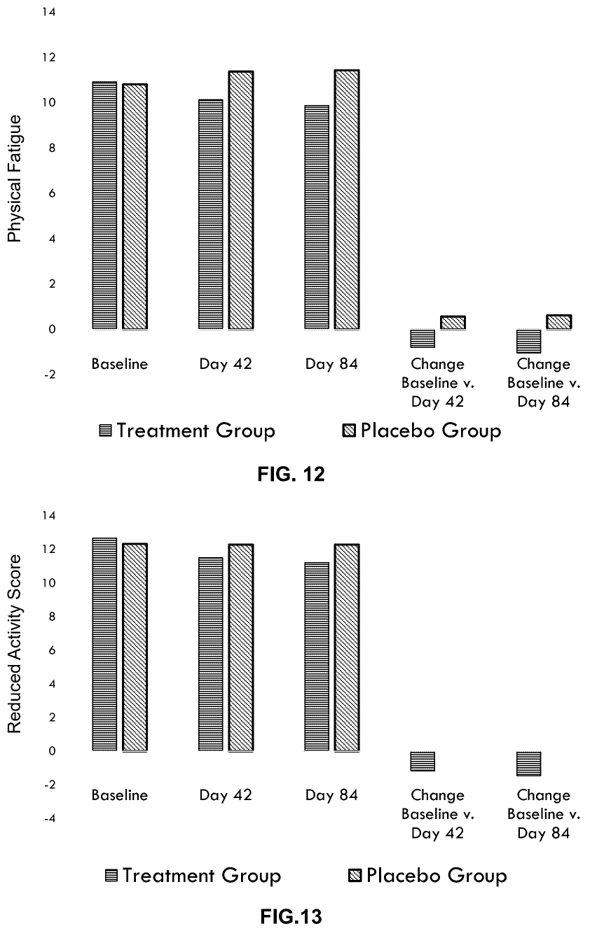

In accordance with the present disclosure, FIG. 12 illustrates a clustered column graph that describes the changes to the physical fatigue-related scores of the human subjects on day 0 (baseline), day 42, and day 84 of the clinical study. In FIG. 12, the changes to the physical fatigue-related scores are plotted on the vertical axis (y-axis), and the days on which the physical fatigue-related scores were determined are indicated on the horizontal axis (x-axis). In accordance with the present disclosure, FIG. 12 is a graphical representation of the physical fatigue-related scores illustrated in Table 12.

In accordance with the present disclosure, Table 13, provided below, illustrates the changes in the reduced activity score observed for each of the subjects in the treatment group and placebo group during the time period (eighty-four days) when the clinical trial was conducted. The reduced activity score is typically used to measure the impact of fatigue on a person's physical, daily, and social activities. A higher score indicates a greater impact of fatigue on various activities, and a lower score indicates a lower impact of fatigue on various activities and points to the alleviation of fatigue.

As illustrated in Table 13, the ‘mean±SD’ values of the treatment group pointed to a significantly lower score, thereby indicating that the subjects in the treatment group were less fatigued than the subjects in the placebo group that did not receive the herbal composition. As per the ‘mean±SD’ values specified in Table 13, the subjects in the treatment group exhibited lesser reductions in activity levels and lower levels of fatigue in comparison to the subjects in the placebo group.

TABLE 13
Reduced Activity Score

| | |
|---|---|
| All the values are | Reduced Activity Score |

| represented as | Herbal Composition | |
|---|---|---|
| ‘mean ± SD’ | (Treatment Group) | Placebo Group |
| Baseline | 12.67 ± 1.24 | 12.33 ± 1.35 |
| Day 42 | 11.50 ± 1.10 | 12.29 ± 1.35 |
| Day 84 | 11.21 ± 1.38 | 12.29 ± 1.35 |
| Change | −1.17 ± 1.09 | 0.0 |
| Baseline vs. Day 42 | | |
| Change | −1.46 ± 1.38 | 0.0 |
| Baseline vs. Day 84 | | |

From the reduced activity score-related changes tabulated in Table 13, it is apparent that the herbal composition causes a significant reduction in the reduced activity score for the subjects in the treatment group (baseline versus day 42: −1.17±1.09 and baseline versus day 84: −1.46±1.38) who were administered the herbal composition daily for sixty days. In fact, the changes to the reduced activity score (on days 42 and 84) for the subjects in the treatment group are denoted by negative values, which, in turn, imply a significant reduction in the reduced activity score, lower levels of fatigue, and very little reduction in the activity levels. Further, as illustrated in Table 13, the reduced activity score for the subjects in the placebo group was null, thereby suggesting no improvement to the activity levels exhibited by the subjects in the placebo group. However, since the changes to the reduced activity score (on days 42 and 84) for the subjects in the treatment group are denoted by negative values, it is apparent that the herbal composition envisaged by the present disclosure brings about a significant reduction in the reduced activity score, thereby implying a reasonable reduction in fatigue levels and activity levels for the subjects who were administered the prescribed dosage of the herbal composition for sixty days.

In accordance with the present disclosure, FIG. 13 illustrates a clustered column graph that describes the changes to the reduced activity scores of the human subjects on day 0 (baseline), day 42, and day 84 of the clinical study. In FIG. 13, the changes to the reduced activity scores are plotted on the vertical axis (y-axis), and the days on which the reduced activity scores were determined are indicated on the horizontal axis (x-axis). In accordance with the present disclosure, FIG. 13 is a graphical representation of the reduced activity scores illustrated in Table 13.

In accordance with the present disclosure, Table 14, provided below, illustrates the changes in the reduced motivation score observed for each of the subjects in the treatment group and placebo group during the time period (eighty-four days) when the clinical trial was conducted. The reduced motivation score is typically used to measure the impact of fatigue on a person's willingness to initiate and persist with activities. A higher score indicates a greater level of reduction in motivation due to fatigue, and a lower score indicates lower levels of reduction in motivation, a higher inclination towards initiating and persisting with various activities, and points to the alleviation of fatigue.

TABLE 14
Reduced Motivation Score

| | |
|---|---|
| All the values are | Reduced Motivation Score |

| represented as | Herbal Composition | |
|---|---|---|
| ‘mean ± SD’ | (Treatment Group) | Placebo Group |
| Baseline | 10.71 ± 1.23 | 10.24 ± 1.29 |
| Day 42 | 10.54 ± 1.14 | 10.24 ± 1.36 |
| Day 84 | 10.04 ± 1.08 | 10.24 ± 1.43 |
| Change | −0.17 ± 0.38 | 0.00 |
| Baseline vs. Day 42 | p−value = 0.0719 | |
| Change | −0.67 ± 0.82 | 0.00 |
| Baseline vs. Day 84 | p−value = 0.0015 | |

From the reduced motivation score-related changes tabulated in Table 14, it is apparent that the herbal composition causes a significant reduction in the reduced motivation score for the subjects in the treatment group (baseline versus day 42: −0.17±0.38 and baseline versus day 84: −0.67±0.82) who were administered the herbal composition daily for sixty days. In fact, the changes in the reduced motivation score (on days 42 and 84) for the subjects in the treatment group are denoted by negative values, which, in turn, imply a significant reduction in the reduced motivation score, lower levels of fatigue, and very little reduction in motivation (for the subjects in the treatment group).

Further, as illustrated in Table 14, the reduced motivation score for the subjects in the placebo group remained unchanged on days 0, 42, and 84. From Table 14, it is apparent that the herbal composition envisaged by the present disclosure brings about a significant reduction in the reduced motivation score for the subjects in the treatment group, thereby implying a reasonable reduction in fatigue levels and a reasonable enhancement in motivation levels that would enable the subjects in the treatment group to initiate and persist with various activities with an improved vigour.

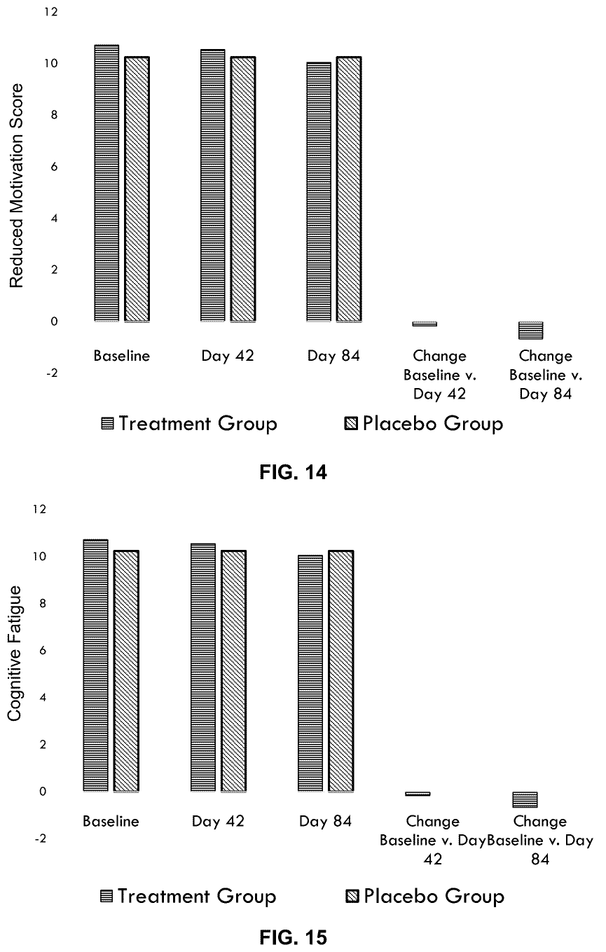

In accordance with the present disclosure, FIG. 14 illustrates a clustered column graph that describes the changes to the reduced motivation scores of the human subjects on day 0 (baseline), day 42, and day 84 of the clinical study. In FIG. 14, the changes to the reduced motivation scores are plotted on the vertical axis (y-axis), and the days on which the reduced motivation scores were determined are indicated on the horizontal axis (x-axis). In accordance with the present disclosure, FIG. 14 is a graphical representation of the reduced motivation scores illustrated in Table 14.

In accordance with the present disclosure, Table 15, provided below, illustrates the changes in the cognitive fatigue scores observed for each of the subjects in the treatment group and placebo group during the time period (eighty-four days) when the clinical trial was conducted.

TABLE 15
Cognitive Fatigue Score

| | |
|---|---|
| All the values are | Cognitive Fatigue |

| represented as | Herbal Composition | |
|---|---|---|
| ‘mean ± SD’ | (Treatment Group) | Placebo Group |
| Baseline | 12.42 ± 0.83 | 12.52 ± 0.87 |
| Day 42 | 12.17 ± 1.09 | 12.33 ± 0.73 |
| Day 84 | 11.46 ± 1.25 | 12.38 ± 0.74 |
| Change | −0.25 ± 0.68 | −0.19 ± 0.60 |
| Baseline vs. Day 42 | | |
| Change | −0.96 ± 1.12 | −0.14 ± 0.66 |
| Baseline vs. Day 84 | | |

As illustrated in Table 15, the ‘mean±SD’ values of the treatment group showed a reasonable decrease (reduction) in the cognitive fatigue-related scores vis-à-vis the placebo group that was not administered the herbal composition, thereby implying a significant reduction in the cognitive fatigue levels of the subjects who were administered the prescribed dose of the herbal composition, and confirming the therapeutic efficacy of the herbal composition in terms of alleviating and treating cognitive fatigue-related symptoms.

From the cognitive fatigue score-related changes tabulated in Table 15, it is apparent that the herbal composition causes a substantial reduction in the cognitive fatigue score for the subjects in the treatment group (baseline versus day 42: −0.25±0.68 and baseline versus day 84: −0.96±1.12) who were administered the herbal composition daily for sixty days. Further, as illustrated in Table 15, the subjects in the placebo group also witnessed a decrease in the cognitive fatigue score on day 42 (−0.19±0.60) and yet another decrease in the cognitive fatigue score on day 84 (−0.14±0.66).

It is pertinent to note that on day 84, the subjects belonging to the treatment group witnessed a significant decrease in the cognitive fatigue score (treatment group—baseline versus day 84: −0.96±1.12), and the subjects in the placebo group witnessed a comparatively lower decrease in the cognitive fatigue score (placebo group—baseline versus day 84: −0.14±0.66). Therefore, from Table 15, it is apparent that the herbal composition envisaged by the present disclosure brings about a significant reduction in the cognitive fatigue score for the subjects in the treatment group, thereby implying a reasonable reduction in cognitive fatigue levels for the subjects who were administered the prescribed dosage of the herbal composition for sixty days.

In accordance with the present disclosure, FIG. 15 illustrates a clustered column graph that describes the changes to the cognitive fatigue-related scores corresponding to the human subjects on day 0 (baseline), day 42, and day 84 of the clinical study. In FIG. 15, the changes to the cognitive fatigue-related scores are plotted on the vertical axis (y-axis), and the days on which the cognitive fatigue-related scores were determined are indicated on the horizontal axis (x-axis). In accordance with the present disclosure, FIG. 15 is a graphical representation of the cognitive fatigue-related scores illustrated in Table 15.

In accordance with the present disclosure, Table 16, provided below, illustrates the changes in the total fatigue scores observed for each of the subjects in the treatment group and placebo group during the time period (eighty-four days) when the clinical trial was conducted.

As illustrated in Table 16, the ‘mean±SD’ values of the treatment group showed a reasonable decrease (reduction) in the total fatigue-related score vis-à-vis the placebo group that was not administered the herbal composition, thereby implying a significant reduction in the total fatigue levels of the subjects who were administered the prescribed dose of the herbal composition, and confirming the therapeutic efficacy of the herbal composition in terms of alleviating and treating and alleviating the total fatigue-related symptoms.

TABLE 16
Total Fatigue Score

| | |
|---|---|
| All the values are | Total Fatigue |

| represented as | Herbal Composition | |
|---|---|---|
| ‘mean ± SD’ | (Treatment Group) | Placebo Group |
| Baseline | 58.58 ± 2.67 | 57.71 ± 2.70 |
| Day 42 | 55.00 ± 2.62 | 58.05 ± 3.06 |
| Day 84 | 53.04 ± 2.90 | 58.19 ± 2.96 |
| Change | −3.58 ± 2.32 | 0.33 ± 1.39 |
| Baseline vs. Day 42 | | |
| Change | −5.54 ± 2.62 | 0.48 ± 1.29 |
| Baseline vs. Day 84 | | |

From the cognitive fatigue score-related changes tabulated in Table 16, it is apparent that the herbal composition causes a substantial reduction in the total fatigue score for the subjects in the treatment group (baseline versus day 42: −3.58±2.32 and baseline versus day 84: −5.54±2.62) who were administered the herbal composition daily for sixty days. Further, as illustrated in Table 16, the subjects in the placebo group witnessed a marginal increase in the total fatigue score on day 42 (0.33±1.39) and yet another marginal increase in the total fatigue score on day 84 (0.62±0.97).

It is pertinent to note that while the subjects belonging to the treatment group witnessed a significant decrease in the total fatigue score (treatment group—baseline versus day 42: −3.58±2.32 and baseline versus day 84: −5.54±2.62), with the total fatigue score for the treatment group being represented by negative values for days 42 and 84, the subjects in the placebo group witnessed no such significant reduction but a marginal increase in the total fatigue scores (for days 42 and 84). Therefore, from Table 16, it is apparent that the herbal composition envisaged by the present disclosure brings about a reduction in the total fatigue score, thereby implying a reasonable reduction in total fatigue levels for the subjects who were administered the prescribed dosage of the herbal composition for sixty days.

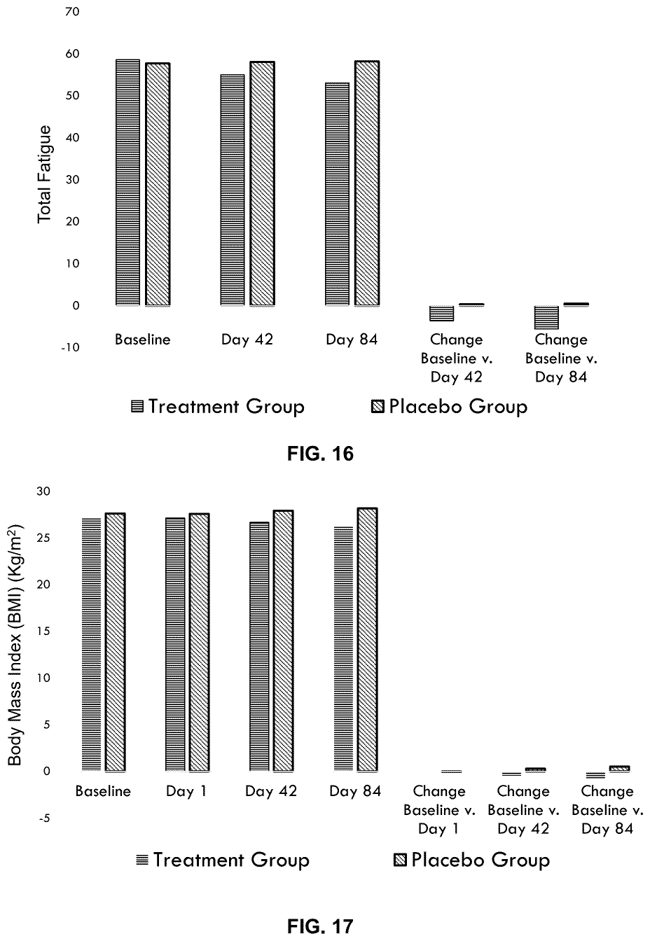

In accordance with the present disclosure, FIG. 16 illustrates a clustered column graph that describes the changes to the total fatigue-related scores corresponding to the human subjects on day 0 (baseline), day 42, and day 84 of the clinical study. In FIG. 16, the changes to the total fatigue-related scores are plotted on the vertical axis (y-axis), and the days on which the total fatigue-related scores were determined are indicated on the horizontal axis (x-axis). In accordance with the present disclosure, FIG. 16 is a graphical representation of the total fatigue-related scores illustrated in Table 16.

In accordance with the present disclosure, Table 17, provided below, illustrates the changes in the Body Mass Index (BMI) observed for each of the subjects in the treatment group and placebo group during the time period (eighty-four days) when the clinical trial was conducted. As illustrated in Table 17, the ‘mean±SD’ values of the treatment group showed a reasonable decrease (reduction) in the Body Mass Index (BMI) vis-à-vis the placebo group that was not administered the herbal composition, thereby implying a significant reduction in the BMI of the subjects who were administered the prescribed dose of the herbal composition, and confirming the therapeutic efficacy of the herbal composition in terms of reducing the BMI and addressing the issue of abnormal weight gain.

TABLE 17
Changes to the Body Mass Index (BMI)

| | |
|---|---|
| All the values are | Body Mass Index (BMI) (Kg/m²) |

| represented as | Herbal Composition | |
|---|---|---|
| ‘mean ± SD’ | (Treatment Group) | Placebo Group |
| Baseline | 27.14 ± 1.55 | 27.63 ± 1.47 |
| Day 1 | 27.14 ± 1.55 | 27.60 ± 1.45 |
| Day 42 | 26.68 ± 1.67 | 27.93 ± 1.62 |
| Day 84 | 26.31 ± 1.77 | 28.16 ± 1.70 |
| Change | 0.00 ± 0.05 | −0.03 ± 0.11 |
| Baseline vs. Day 1 | | |
| Change | −0.46 ± 0.35 | 0.30 ± 0.44 |
| Baseline vs. Day 42 | | |
| Change | −0.83 ± 0.59 | 0.53 ± 0.59 |
| Baseline vs. Day 84 | | |

From the BMI-related changes tabulated in Table 17, it is apparent that the herbal composition causes a substantial reduction in the BMI for the subjects in the treatment group (baseline versus day 42: −0.46±0.35 and baseline versus day 84: −0.83±0.59) who were administered the herbal composition daily for sixty days. Further, as illustrated in Table 17, the subjects in the placebo group witnessed a marginal increase in the BMI on day 42 (0.30±0.44) and yet another marginal increase in the BMI on day 84 (0.53±0.59).

It is pertinent to note that while the subjects belonging to the treatment group witnessed a significant decrease in the BMI on days 42 and 84 (treatment group—baseline versus day 42: −0.46±0.35 and baseline versus day 84: −0.83±0.59), with the change in the BMI for the subjects of the treatment group being represented by negative values for days 42 and 84, the subjects in the placebo group witnessed no such significant reduction but a marginal increase in the BMI (for days 42 and 84). Therefore, from Table 17, it is apparent that the herbal composition envisaged by the present disclosure brings about a reduction in the BMI, thereby implying a reasonable reduction in abnormal weight gain in the subjects who were administered the prescribed dosage of the herbal composition for sixty days.

In accordance with the present disclosure, FIG. 17 illustrates a clustered column graph that describes the changes to the body mass index (BMI) of the human subjects on day 0 (baseline), day 42, and day 84 of the clinical study. In FIG. 17, the changes to the body mass index are plotted on the vertical axis (y-axis), and the days on which the body mass index-related scores were determined are indicated on the horizontal axis (x-axis). In accordance with the present disclosure, FIG. 17 is a graphical representation of the body mass index-related scores illustrated in Table 17.

In accordance with the present disclosure, Table 18, provided below, illustrates the changes in the body weight observed for each of the subjects in the treatment group and placebo group during the time period (eighty-four days) when the clinical trial was conducted.

TABLE 18
Changes to the Body Weight

| | |
|---|---|
| All the values are | Body Weight (Kg) |

| represented as | Herbal Composition | |
|---|---|---|
| ‘mean ± SD’ | (Treatment Group) | Placebo Group |
| Baseline | 73.70 ± 1.55 | 73.46 ± 1.47 |
| Day 1 | 73.70 ± 1.55 | 73.38 ± 1.45 |
| Day 42 | 72.43 ± 1.67 | 74.24 ± 1.62 |
| Day 84 | 71.36 ± 1.77 | 74.85 ± 1.70 |
| Change | 0.00 ± 0.09 | −0.08 ± 0.32 |
| Baseline vs. Day 1 | | |
| Change | −1.27 ± 0.90 | 0.78 ± 1.12 |
| Baseline vs. Day 42 | | |
| Change | −2.34 ± 1.63 | 1.39 ± 1.49 |
| Baseline vs. Day 84 | | |

As illustrated in Table 18, the ‘mean±SD’ values of the treatment group showed a reasonable decrease (reduction) in the Body Mass Index (BMI) vis-à-vis the placebo group that was not administered the herbal composition, thereby implying a significant reduction in the BMI of the subjects who were administered the prescribed dose of the herbal composition, and confirming the therapeutic efficacy of the herbal composition in terms of reducing the BMI and addressing the issue of abnormal weight gain.

From the body weight-related changes tabulated in Table 18, it is apparent that the herbal composition causes a substantial reduction in the body weight for the subjects in the treatment group (baseline versus day 42: −1.27±0.90 and baseline versus day 84: −2.34±1.63) who were administered the herbal composition daily for sixty days. Further, as illustrated in Table 18, the subjects in the placebo group witnessed a marginal increase in the BMI on day 42 (0.78±1.12) and a significant increase in the BMI on day 84 (1.39±1.49).

It is pertinent to note that while the subjects belonging to the treatment group witnessed a significant decrease in the body weight on days 42 and 84 (treatment group—baseline versus day 42: −1.27±0.90 and baseline versus day 84: −2.34±1.63), with the changes in the body weight for the treatment group being represented by negative values for days 42 and 84, the subjects in the placebo group witnessed no such significant reduction but an increase in the body weight (for days 42 and 84). Therefore, from Table 18, it is apparent that the herbal composition envisaged by the present disclosure brings about a reduction in body weight, thereby implying a reasonable reduction in abnormal weight gain for the subjects who were administered the prescribed dosage of the herbal composition for sixty days.

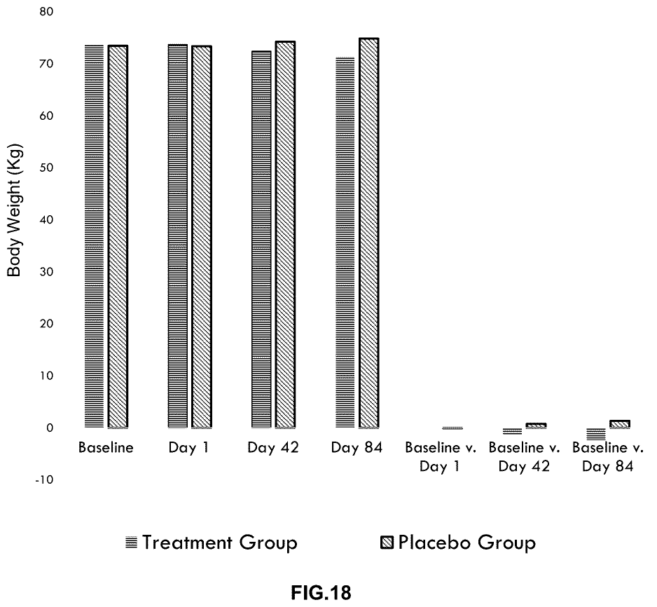

In accordance with the present disclosure, FIG. 18 illustrates a clustered column graph that describes the changes to the body weight of the human subjects on day 0 (baseline), day 42, and day 84 of the clinical study. In FIG. 18, the changes to the body weight are plotted on the vertical axis (y-axis), and the days on which the body weight-related readings were taken are indicated on the horizontal axis (x-axis). In accordance with the present disclosure, FIG. 18 is a graphical representation of the body weight-related readings illustrated in Table 18.

In accordance with the present disclosure, the herbal composition, when administered to human beings as per the prescribed dosage (500 mg) for at least sixty consecutive days, reduces the accumulation of adipose tissue, the total cholesterol levels, triglyceride levels, and low-density lipoprotein (LDL) cholesterol levels, and elevates (enhances) the high-density lipoprotein (HDL) cholesterol levels. Further, the herbal composition also modulates the tumour necrosis factor-alpha (TNF-α) levels, adiponectin levels, and interleukin-6 (IL-6) levels. The herbal composition also causes a decrease in the organ weight, especially liver weight, and modulates the liver function test (LFT)-related parameters, including alkaline phosphatase levels, Serum Glutamic-Oxaloacetic Transaminase (SGOT) levels, and Serum Glutamic-Pyruvic Transaminase (SGPT) levels, thereby promoting improved liver health. Further, the herbal composition brings about an improvement in the very-low-density lipoprotein (VLDL) cholesterol levels, total cholesterol-HDL cholesterol ratio, and LDL cholesterol-HDL cholesterol ratio. The herbal composition also improves glucose uptake (i.e., blood glucose metabolism), thereby improving insulin sensitivity and modulating insulin resistance. Further, by improving insulin sensitivity and modulating insulin resistance, the herbal composition improves the human body's response to insulin and therefore treats and alleviates the symptoms associated with pre-diabetes and type 2 diabetes.

The herbal composition also causes a reduction in the body fat percentage, hip circumference, and waist circumference, enhances lean body mass, and brings about an improvement in the waist-to-hip ratio, thereby treating and alleviating central obesity. Further, the herbal composition effectively treats and alleviates the symptoms associated with general fatigue, physical fatigue, motivation fatigue, and cognitive fatigue, in addition to treating and alleviating the symptoms associated with stress-induced fatigue. The herbal composition also causes a significant reduction in body weight and significantly improves the body mass index (BMI), thereby facilitating effective weight management and treating and alleviating central obesity.

### TECHNICAL ADVANTAGES

The technical advantages envisaged by the present disclosure include the realization of a herbal composition that is highly effective against a multitude of metabolic disorders plaguing mammals, especially human beings, including central obesity, dyslipidemia, stress-induced fatigue, pre-diabetes, higher blood sugar levels, and higher blood pressure levels.

The herbal composition envisaged by the present disclosure, when consumed as per the prescribed dosage, reduces bodily inflammation and also brings about a decrease in body mass. The herbal composition envisaged by the present disclosure also promotes weight loss, improved energy recovery and retention, lower blood sugar levels, improved blood pressure levels, and improved lipid profile. The herbal composition envisaged by the present disclosure is safe to use, designed to be well-tolerable to the human body, does not cause addiction even after extended use, and is free of harmful side effects.

The herbal composition, when consumed as per the prescribed dosage, promotes sustained weight reduction without the need for an additional exercise regimen and diet-related restrictions. The herbal composition envisaged by the present disclosure contains standardized herbal extracts and is adapted to reduce the accumulation of adipose tissue in human beings suffering from central obesity. Furthermore, the herbal composition, when consumed as per the prescribed dosage, also triggers a decrease in the cholesterol levels, triglyceride levels, and low-density lipoprotein levels, all three indicative of improved heart health and reduced risk of cardiovascular diseases. Subsequently, the herbal composition also brings about an elevation in the high-density lipoprotein (HDL) levels, with higher HDL levels indicating reduced plaque formation in the blood-carrying arteries and improved heart health.

The herbal composition envisaged by the present disclosure also brings about a reduction in the levels of tumor necrosis factor-alpha (TNF-α) and prevents the over-production of tumor necrosis factor-alpha, a crucial cytokine that, when over-produced, promotes inflammation and acts as a contributory factor to major autoimmune and inflammatory diseases such as rheumatoid arthritis and Crohn's disease. Further, the herbal composition, when consumed as per the prescribed dosage, also enhances the adiponectin levels, with the enhanced adiponectin levels alleviating central obesity, promoting improved metabolic health, and reducing the risk of cardiovascular diseases.

Further, the herbal composition, when consumed as per the prescribed dosage, also modulates the interleukin-6 (IL-6) levels, which, in turn, act as an inflammatory marker, with higher levels of interleukin-6 indicating inflammation and a possible infection. The herbal composition, by modulating the levels of interleukin-6 (IL-6), alleviates the symptoms associated with cardiovascular diseases, rheumatoid arthritis, lupus, stroke, and type 2 diabetes. Further, the herbal composition, when consumed as per the prescribed dosage, also brings about a decrease in the levels of biomarkers, viz., bilirubin, albumin time, prothrombin time, Aspartate Aminotransferase (AST), Alanine Aminotransferase (ALT), Alanine Transaminase, Aspartate Transaminase, Alkaline Phosphatase, and Gamma-Glutamyl Transferase, typically used for liver function tests (LFT) and for indicating abnormalities in liver function, and specifically, liver damage.

Further, the herbal composition, when consumed as per the prescribed dosage, brings about a significant decrease in the body weight of human beings. Further, the herbal composition also brings about a significant decrease in the organ weight, especially the liver weight. Further, the herbal composition also brings about a significant decrease in the epididymal fat, which serves as a fat storage (fat depot) in human beings. Further, the herbal composition also brings about a significant decrease in the subcutaneous fat, which is located just below the skin (epidermal layer). The herbal composition, when consumed as per the prescribed dosage, promotes sustained weight reduction without the need for an additional exercise regimen and diet-related restrictions. The herbal composition, when consumed as per the prescribed dosage, modifies a multitude of metabolic pathways that support fat loss and energy recovery. Specifically, the herbal composition inhibits Monoacylglycerol Acyltransferase 2 (MGAT2) enzyme, which, in turn, synthesizes triglycerides, thereby reducing triglyceride levels in the human body and lowering fat absorption, and thereby alleviating and treating the symptoms associated with central obesity and type 2 diabetes. Furthermore, the herbal composition acts as a glucagon-like peptide-1 (GLP-1) receptor agonist and mimics the actions of GLP-1, a natural gut hormone, thereby lowering blood sugar levels by enhancing insulin secretion and reducing glucagon release. Further, by acting as a GLP-1 receptor agonist, the herbal composition also controls the appetite and slows down the process of digestion, thereby promoting weight loss and improvements in lipid profile and blood pressure levels.

As used herein, “about” or “approximately” shall generally mean within 20 percent of a given value or range. Other than in the operating examples, or where otherwise indicated, all numbers expressing quantities of ingredients and/or reaction conditions are to be understood as being modified in all instances by the term “about”.

We claim:

1. A method for treating a metabolic disorder in a mammal, the method comprising administering to the mammal a therapeutically effective amount of a herbal composition, said herbal composition comprising:
an extract of Dolichos lablab in an amount ranging between 0.1% to 57.38% by weight of said herbal composition;
an extract of Achyranthes aspera in an amount ranging between 0.1% to 32.79% by weight of said herbal composition; and
an extract of at least one of Cinnamomum zeylanicum and Cinnamomum cassia in an amount ranging between 0.1% to 9.83% by weight of said herbal composition;
wherein said metabolic disorder is selected from a group of metabolic disorders consisting of dyslipidemia, stress-induced fatigue, pre-diabetes, type 2 diabetes, high blood sugar levels, elevated blood pressure levels, and abdominal obesity; and
wherein said herbal composition, when administered in said therapeutically effective amount to said mammal, causes an enhancement of at least one of adiponectin levels and high-density lipoprotein (HDL) levels in said mammal, and a reduction in at least one of accumulation of adipose tissue, total cholesterol levels, triglyceride levels, and low-density lipoprotein (LDL) levels in said mammal; and
wherein said herbal composition, when administered in said therapeutically effective amount to said mammal, exhibits enhanced activity at the glucagon-like peptide-1 (GLP-1) receptor in comparison to native glucagon, and at least partially and selectively inhibits Monoacylglycerol Acyltransferase 2 (MGAT2) enzyme.

2. The method as claimed in claim 1, wherein:
the extract of Dolichos lablab is prepared by treating plant parts of Dolichos lablab with a first solvent mixture at a temperature ranging between 60 and 65 degree Celsius, said first solvent mixture having a pH value ranging between 4 and 7, said first solvent mixture containing ethanol in an amount ranging between 60% to 65% by weight of said first solvent mixture and water in an amount ranging between 35% to 40% by weight of said first solvent mixture;
the extract of Achyranthes aspera is prepared by treating plant parts of Achyranthes Aspera with a second solvent mixture at a temperature ranging between 55 and 60 degree Celsius, said second solvent mixture having a pH value ranging between 4 and 7, said second solvent mixture containing ethanol in an amount ranging between 50% to 55% by weight of said second solvent mixture and water in an amount ranging between 45% to 50% by weight of said second solvent mixture; and
the extract of at least one of Cinnamomum zeylanicum and Cinnamomum cassia is prepared by treating plant parts of at least one of Cinnamomum zeylanicum and Cinnamomum cassia with a third solvent mixture at a temperature ranging between 55 and 60 degree Celsius, said third solvent mixture having a pH value ranging between 3 and 5, said third solvent mixture containing ethanol in an amount ranging between 60% to 65% by weight of said third solvent mixture and water in an amount ranging between 35% to 40% by weight of said third solvent mixture.

3. The method as claimed in claim 1, wherein said herbal composition is standardized to contain:
proanthocyanidins in a concentration ranging between 2% and 20% by weight of the herbal composition, and wherein the concentration of the proanthocyanidins is not less than 2%;
saponins in a concentration ranging between 15% and 50% by weight of the herbal composition, and wherein the concentration of the saponins is not less than 15%;
polyphenols in a concentration ranging between 3% and 10% by weight of the herbal composition, and wherein the concentration of polyphenols is not less than 2%; and
tannins in a concentration ranging between 2% and 20% by weight of the herbal composition, and wherein the concentration of the tannins is not less than 3%.

4. The method as claimed in claim 2, wherein the herbal composition comprises at least one of fractions, active compounds, and phytochemicals derived from the plant parts of Dolichos lablab, Achyranthes Aspera, Cinnamomum zeylanicum, and Cinnamomum Cassia.

5. The method as claimed in claim 4, wherein the plant parts are selected from a group consisting of leaves, stems, tender stems, tender twigs, aerial parts, whole fruits, fruit rinds, seeds, roots, bark, hardwood, and combinations thereof.

6. The method as claimed in claim 2, wherein:
said first solvent mixture contains at least one of ketones, chlorinated hydrocarbons, C1-C7 hydrocarbons, and esters in an amount ranging between 60% to 65% by weight of said first solvent mixture, and the water in an amount ranging between 35% to 40% by weight of said first solvent mixture;
said second solvent mixture contains at least one of the ketones, the chlorinated hydrocarbons, the C1-C7 hydrocarbons, and the esters in an amount ranging between 50% to 55% by weight of said second solvent mixture, and the water in an amount ranging between 45% to 50% by weight of said second solvent mixture; and
said third solvent mixture contains at least one of the ketones, the chlorinated hydrocarbons, the C1-C7 hydrocarbons, and the esters in an amount ranging between 60% to 65% by weight of said third solvent mixture, and the water in an amount ranging between 35% to 40% by weight of said third solvent mixture.

7. The method as claimed in claim 1, wherein the herbal composition comprises at least one additional component selected from a group consisting of pharmaceutically acceptable active ingredients, vitamins, minerals, excipients, carriers, and diluents, and wherein said at least one additional component is present in said herbal composition in an amount ranging between 1% to 10% by weight of said herbal composition.

8. The method as claimed in claim 6, wherein:
the C1-C7 hydrocarbons are selected from a group of hydrocarbons consisting of methane, ethane, propane, butane, pentane, hexane, and heptane;
the ketones include at least one of acetone and methyl isobutyl ketone; and
the chlorinated hydrocarbons include at least one of methylene dichloride and chloroform.

9. The method as claimed in claim 1, wherein the mammal is a human being.

10. A method for treating a metabolic disorder in a mammal, the method comprising administering to the mammal a therapeutically effective amount of a herbal composition, said herbal composition comprising:
an extract of Dolichos lablab in an amount ranging between 0.1% to 57.38% by weight of said herbal composition;
an extract of Achyranthes aspera in an amount ranging between 0.1% to 32.79% by weight of said herbal composition; and
an extract of at least one of Cinnamomum zeylanicum and Cinnamomum cassia in an amount ranging between 0.1% to 9.83% by weight of said herbal composition; and
wherein said herbal composition, when administered in said therapeutically effective amount to said mammal, alleviates physical and cognitive fatigue induced by stress in said mammal; and
wherein said herbal composition, when administered in said therapeutically effective amount to said mammal, treats at least one metabolic disorder selected from a group consisting of dyslipidemia, pre-diabetes, type 2 diabetes, high blood sugar levels, elevated blood pressure levels, and abdominal obesity.

11. A herbal composition, said herbal composition comprising:
an extract of Dolichos lablab in an amount ranging between 0.1% to 57.38% by weight of said herbal composition;
an extract of Achyranthes aspera in an amount ranging between 0.1% to 32.79% by weight of said herbal composition; and
an extract of at least one of Cinnamomum zeylanicum and Cinnamomum cassia in an amount ranging between 0.1% to 9.83% by weight of said herbal composition; and
wherein said herbal composition when administered in a therapeutically effective amount to a mammal in need thereof treats at least one metabolic disorder selected from a group consisting of dyslipidemia, stress-induced fatigue, pre-diabetes, type 2 diabetes, high blood sugar levels, elevated blood pressure levels, and abdominal obesity, by modulating a plurality of metabolic biomarkers in said mammal, said metabolic biomarkers selected from a group consisting of adiponectin, high-density lipoprotein (HDL), Interleukin-6 (IL-6), total cholesterol, triglycerides, and low-density lipoprotein (LDL).

12. The herbal composition as claimed in claim 11, wherein the herbal composition is standardized to contain:
proanthocyanidins in a concentration ranging between 2% and 20% by weight of the herbal composition, and wherein the concentration of the proanthocyanidins is not less than 2%;
saponins in a concentration ranging between 15% and 50% by weight of the herbal composition, and wherein the concentration of the saponins is not less than 15%;
polyphenols in a concentration ranging between 3% and 10% by weight of the herbal composition, and wherein the concentration of polyphenols is not less than 2%; and
tannins in a concentration ranging between 2% and 20% by weight of the herbal composition, and wherein the concentration of the tannins is not less than 3%.

13. The herbal composition as claimed in claim 11, wherein the herbal composition comprises at least one of fractions, active compounds, and phytochemicals derived from plant parts of Dolichos lablab, Achyranthes Aspera, Cinnamomum zeylanicum, and Cinnamomum Cassia.

14. The herbal composition as claimed in claim 11, wherein the herbal composition comprises at least one additional component selected from a group consisting of pharmaceutically acceptable active ingredients, vitamins, minerals, excipients, carriers, and diluents, and wherein said at least one additional component is present in said herbal composition in an amount ranging between 1% to 10% by weight of said herbal composition.

15. The herbal composition as claimed in claim 11, wherein said herbal composition is formulated as an oral dosage form, and wherein said oral dosage form is selected from a group consisting of a tablet, capsule, soluble powder, chewable tablet, chewable gum, liquefied beverage, and chewable candy.

16. The herbal composition as claimed in claim 11, wherein said herbal composition when administered in said therapeutically effective amount to said mammal elevates at least one of adiponectin levels, and high-density lipoprotein (HDL) levels in said mammal, and decreases at least one of accumulation of adipose tissue, total cholesterol levels, triglycerides levels, and low-density lipoprotein (LDL) levels in said mammal.

17. The herbal composition as claimed in claim 11, wherein said herbal composition, when administered in said therapeutically effective amount to said mammal, exhibits enhanced activity at the glucagon-like peptide-1 (GLP-1) receptor in comparison to native glucagon, and at least partially and selectively inhibits Monoacylglycerol Acyltransferase 2 (MGAT2) enzyme.
